SEQUENCE LISTING (4 sequences)

SEQ ID NO: 1 - protein, 470 aa, synthetic construct
EVQLVESGGGLIQPGGSLRLSCAASGIIVSSNYMTWVRQAPGKGLEWVSVIYSGGSTFYADSVKGRFTISRDNSKNTLYLQMSSLRAEDTAVYYCARDLGPYGMDVWGQGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 214 aa, synthetic construct
DIQLTQSPSFLSASVGDRVTITCRASQGISSDLAWYQQKPGKAPNLLIYAASTLQSGVPSRFSGSGSGTEFTLTISSLQPEDFATYYCQQLNSDLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 4 - protein, 585 aa, synthetic construct
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

(19) United States
(12) Patent Application Publication
(10) Pub. No.: US 2024/0209065 A1
(43) Pub. Date: Jun. 27, 2024

(54) SECRETORY IGA ANTIBODIES AGAINST COVID INFECTION

(71) Applicants: Qiang Hammarström PAN, Huddinge (SE); Lennart HAMMARSTRÖM, Huddinge (SE); Harold MARCOTTE, Huddinge (SE); Singlomics Beijing DanXu Biopharmaceuticals Co., Ltd., Beijing (CN); Wuhan Institute of Biological Products Co., Ltd., Wuhan (CN)

(72) Inventors: Qiang Hammarström PAN, Huddinge (SE); Lennart HAMMARSTRÖM, Huddinge (SE); Harold MARCOTTE, Huddinge (SE)

(21) Appl. No.: 18/518,425

(22) Filed: Nov. 22, 2023

(30) Foreign Application Priority Data
Dec. 23, 2022 (WO) PCT/CN2022/141339

(51) Int. Cl. C07K 16/10; A61P 31/14

(52) U.S. Cl. CPC C07K 16/1003; A61P 31/14; C07K 2317/76

(57) ABSTRACT

The present disclosure provides an immunoglobulin A neutralizing SARS-CoV-2 Omicron variant, isolated polynucleotides encoding the same, pharmaceutical compositions comprising the same, and the uses thereof.

### CROSS-REFERENCE TO RELATED APPLICATIONS

This application claims priority to PCT/CN2022/141339, filed Dec. 23, 2022, the disclosure of which is incorporated herein by reference.

### SEQUENCE LISTING

This application contains a Sequence Listing which is submitted electronically in XML format and is hereby incorporated by reference in its entirety. Said XML copy, created on Nov. 16, 2023, is named 082218-8001US01. xml and is 5,830 bytes in size.

### FIELD OF THE INVENTION

The present disclosure relates generally to the fields of medicine. More particular, the disclosure relates to antibodies that neutralize to coronavirus (2019-nCoV, also known as SARS-CoV-2) variants and the uses thereof.

### BACKGROUND

As severe acute respiratory syndrome coronavirus 2 (SARS-CoV-2) continues to spread worldwide, selection pressure to evade the antibodies in convalescent and/or vaccinated individuals has led to viral mutations and the emergence of Variants of Concerns (VOCs), such as Alpha (B.1.1.7), Beta (B.1.351), Gamma (P.1) and Delta (B.1.617.2)¹. Mutations on the viral spike (S) protein, including the receptor-binding domain (RBD), may lead to a reduced susceptibility to neutralization antibody, an increased binding to the ACE2 receptor on host cells and a higher transmissibility and infectivity¹. The emergence of the Omicron variant in South Africa in November 2021, and its rapid spread worldwide, has strengthened concerns about vaccine efficacy and antibody therapy due to the large number of mutations in the S protein²⁻⁵. The original Omicron variant (B.1.1.529) already harbors 37 mutations in the S glycoprotein, including 15 in the RBD⁶ and is continuously evolving, and has hitherto divided into five major lineages, BA.1 to BA.5⁷. Novel subvariants such as BA.2.75, BQ.1, XBB, derived from either BA.2 or BA.4/5, have rapidly emerged⁸. The rise of these subvariants seems to stem from their capacity to infect individuals who are immune to earlier Omicron subvariants⁹. Development of novel antibody therapies that remain efficacious despite virus evolution is thus urgently needed.

### BRIEF SUMMARY OF THE INVENTION

The present disclosure provides an immunoglobulin A (IgA) neutralizing SARS-CoV-2 Omicron variant, isolated polynucleotides encoding the same, pharmaceutical compositions comprising the same, and the uses thereof.

In one aspect, the present disclosure provides an IgA antibody which binds to and neutralizes SARS-CoV-2 Omicron variant, the IgA comprising a paired heavy chain and light chain variable domains as disclosed in the references listed in Table 1.

In some embodiments, the IgA antibody disclosed herein comprises a paired heavy chain and light chain variable domains of an antibody selected from the group consisting of casirivimab (Regeneron Pharmaceuticals), imdevimab (Regeneron Pharmaceuticals), etesevimab (Eh Lilly and Company), bamlanivimab (Eh Lilly and Company), CT-P59 (Celltrion Healthcare), BRII-196 (Brii Biosciences), BRII-198 (Brii Biosciences), VIR-7831 (Vir Biotechnology), AZD7442 (AstraZeneca), AZD8895 (AstraZeneca), AZD1061 (AstraZeneca), TY027 (Tychan Pte. Ltd.), SCTA01 (Sinocelltech Ltd.), MW33 (Mabwell Bioscience Co., Ltd.), JS016 (Junshi Biosciences), DXP593 (Singlomics/Beigene), DXP604 (Singlomics/Beigene), STI-2020 (Sorrento Therapeutics), BI 767551/DZIF-lOc (U. Cologne/Boehringer Ingelheim), COR-101 (CORAT Therapeutics), HLX70 (Hengenix Biotech), ADM03820 (Ology Bioservices), HFB30132A (HiFiBiO Therapeutics), ABBV-47D11 (AbbVie), C144-LS (Bristol-Myers Squibb, Rockefeller University), C-135-LS (Bristol-Myers Squibb, Rockefeller University), LY-CovMab (Luye Pharma), JMB2002 (Jemincare), ADG20 (Adagio Therapeutics), LY-Cov1404 (AbCellera; Eh Lilly and Company).

In some embodiments, the IgA antibody disclosed herein is DXP-604 IgA antibody which comprises a heavy chain comprises or consists of the amino acid sequence set forth in SEQ ID NO: 1, and a light chain comprises or consists of the amino acid sequence set forth in SEQ ID NO: 2; or variants thereof wherein the heavy chain and/or light chain has one, two, or three amino acid substitutions, additions, deletions or combination thereof.

In some embodiments, the IgA antibody disclosed herein is dimeric. In some embodiments, the IgA antibody disclosed herein further comprises a J chain comprises or consists of the amino acid sequence set forth in SEQ ID NO: 3, or a sequence variant thereof comprising one, two, or three acid substitutions, additions, deletions or combination thereof.

In some embodiments, the IgA antibody disclosed herein is secretory. In some embodiments, the IgA antibody disclosed herein further comprises a secretory component comprises or consists of the amino acid sequence set forth in SEQ ID NO: 4, or a sequence variant thereof comprising one, two, or three acid substitutions, additions, deletions or combination thereof.

In some embodiments, the IgA antibody disclosed herein is capable of neutralizing a SARS-CoV-2 infection and/or of neutralizing an infection of a target cell with an IC50 of about 20 to about 30 ng/ml. In some embodiments, the IgA antibody disclosed herein is capable of neutralizing a SARS-CoV-2 infection and/or of neutralizing an infection of a target cell with an IC50 of about 10 to about 20 ng/ml. In some embodiments, the IgA antibody disclosed herein is capable of neutralizing a SARS-CoV-2 infection and/or of neutralizing an infection of a target cell with an IC50 of about 5 to about 10 ng/ml. In some embodiments, the IgA antibody disclosed herein is capable of neutralizing a SARS-CoV-2 infection and/or of neutralizing an infection of a target cell with an IC50 of about 1 to about 5 ng/ml.

In another aspect, the present disclosure provides an isolated polynucleotide encoding the IgA antibody disclosed herein. In some embodiments, the polynucleotide disclosed herein comprises deoxyribonucleic acid (DNA) or ribonucleic acid (RNA), wherein the RNA optionally comprises messenger RNA (mRNA). In some embodiments, the polynucleotide disclosed herein is codon-optimized for expression in a host cell.

In another aspect, the present disclosure provides a recombinant vector comprising the polynucleotide disclosed herein.

In another aspect, the present disclosure provides a host cell comprising the polynucleotide disclosed herein and/or the vector disclosed herein, wherein the polynucleotide is heterologous to the host cell.

In another aspect, the present disclosure provides a composition comprising the IgA antibody disclosed herein and a pharmaceutically acceptable excipient, carrier, or diluent.

In another aspect, the present disclosure provides a method for treating infection of SARS-CoV-2 Omicron variant. In some embodiments, the method comprises administering the IgA antibody disclosed herein or the composition disclosed herein to a subject in need thereof. In some embodiments, the IgA antibody or the composition is administered via intranasal.

In another aspect, the present disclosure provides the use of (i) the IgA antibody disclosed herein, (ii) the polynucleotide disclosed herein, (iii) the recombinant vector disclosed herein, (iv) the host cell disclosed herein, and/or (v) the composition disclosed herein, in the manufacture of medicament for preventing or treating a coronavirus infection, e.g. a SARS-CoV-2 Omicron variant infection, in a subject.

### BRIEF DESCRIPTION OF FIGURES

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C show Secretory IgA anti-RBD antibodies are produced at low level following vaccination. Salivary anti-RBD IgA (FIG. 1A), IgG (FIG. 1B) and IgM (FIG. 1C) antibodies in different vaccination groups. For each group, the number of samples (N=) and median antibody titres are shown below the X-axis. Whiskers indicate the interquartile range. The results of anti-RBD antibodies are presented as arbitrary units (AU)/μg total IgA (salivary IgA), binding antibody units (BAU ml-1) (salivary and plasma IgG) or arbitrary Units (AU)/ml (salivary IgM). HV: Heterologous vaccination (two doses inactivated vaccine followed by a heterologous mRNA boost, Inf+Vac: one or two doses mRNA vaccine after SARS-CoV-2 infection, BTI: breakthrough infection. Two-sided Mann-Whitney U test was used. *P<0.05, ** P<0.01, *** P<0.001, and **** P<0.0001. ns, not significant.

FIG. 2 shows antibodies engineered from IgG into monomeric, dimeric and secretory IgA1.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. Definitions

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both element or component comprising one unit and elements or components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable domain (V_{H}) and a constant region including a first, second, and third constant domain (C_{H1}, C_{H2}, C_{H3}, respectively); mammalian light chains are classified as λ or κ, while each light chain consists of a variable domain (V_{L}) and a constant domain (C_{L}). A typical IgG antibody has a “Y” shape, with the stem of the Y typically consisting of the second and third constant domains of two heavy chains bound together via disulfide bonding. Each arm of the Y includes the variable domain and first constant domain of a single heavy chain bound to the variable and constant domains of a single light chain. The variable domains of the light and heavy chains are responsible for antigen binding. The variable domains in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. (1985) 186(3):651-63; Chothia, C. and Lesk, A. M., J. Mol. Biol. (1987) 196:901; Chothia, C. et al., Nature (1989) 342(6252):877-83; Marie-Paule Lefranc et al., Developmental and Comparative Immunology (2003) 27: 55-77; Marie-Paule Lefranc et al., Immunome Research (2005) 1(3); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant domains of the heavy and light chains are not involved in antigen-binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

The term “antigen” refers to a substance capable of inducing adaptive immune responses. Specifically, an antigen is a substance specifically bound by antibodies or T lymphocyte antigen receptors. Antigens are usually proteins and polysaccharides, less frequently also lipids. Suitable antigens include without limitation parts of bacteria (coats, capsules, cell walls, flagella, fimbrai, and toxins), viruses, and other microorganisms. Antigens also include tumor antigens, e.g., antigens generated by mutations in tumors. As used herein, antigens also include immunogens and haptens.

An “Fc” region comprises two heavy chain fragments comprising the C_{H}2 and C_{H}3 domains of an antibody. The two heavy chain fragments are held together by two or more disulfide bonds and by hydrophobic interactions of the C_{H}3 domains. The Fc region of the antibody is responsible for various effector functions such as antibody-dependent cell-mediated cytotoxicity (ADCC), and complement dependent cytotoxicity (CDC), but does not function in antigen binding.

The term “specific binding” or “specifically binds” as used herein refers to a non-random binding reaction between two molecules, such as for example between an antibody and an antigen. In certain embodiments, the antibodies or antigen-binding fragments provided herein specifically bind to human CCR4 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁸ M, ≤5×10⁻⁹ M, ≤4×10⁻⁹M, ≤3×10⁻⁹M, ≤2×10⁻⁹ M, or ≤10⁻⁹ M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The ability to “block binding” or to “compete for the same epitope” as used herein refers to the ability of an antibody or antigen-binding fragment to inhibit the binding interaction between two molecules to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

The term “epitope” as used herein refers to the specific group of atoms or amino acids on an antigen to which an antibody binds. Two antibodies may bind the same or a closely related epitope within an antigen if they exhibit competitive binding for the antigen. For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequence when optimally aligned.

The phrase “host cell” as used herein refers to a cell into which an exogenous polynucleotide and/or a vector has been introduced.

The term “humanized” as used herein means that the antibody or antigen-binding fragment comprises CDRs derived from non-human animals, FR regions derived from human, and when applicable, the constant regions derived from human.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody” refers to the antibody having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

A “leader peptide” or “signal peptide” refers to a peptide having a length of about 5-30 amino acids that is present at the N-terminus of newly synthesized proteins that form part of the secretory pathway. Proteins of the secretory pathway include, but are not limited to proteins that reside either inside certain organelles (the endoplasmic reticulum, Golgi or endosomes), are secreted from the cell, or are inserted into a cellular membrane. In some embodiments, the leader peptide forms part of the transmembrane domain of a protein.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol. (1990) 215:403-410; Stephen F. et al., Nucleic Acids Res. (1997) 25:3389-3402), ClustalW2 (available on the website of European Bioinformatics Institute, see also, Higgins D. G. et al., Methods in Enzymology (1996) 266:383-402; Larkin M. A. et al., Bioinformatics (2007) 23:2947-8), and ALIGN or Megalign (DNASTAR) software. Those skilled in the art may use the default parameters provided by the tool, or may customize the parameters as appropriate for the alignment, such as for example, by selecting a suitable algorithm.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “polypeptide” or “protein” means a string of at least two amino acids linked to one another by peptide bonds. Polypeptides and proteins may include moieties in addition to amino acids (e.g., may be glycosylated) and/or may be otherwise processed or modified. Those of ordinary skill in the art will appreciate that a “polypeptide” or “protein” can be a complete polypeptide chain as produced by a cell (with or without a signal sequence), or can be a functional portion thereof. Those of ordinary skill will further appreciate that a polypeptide or protein can sometimes include more than one polypeptide chain, for example linked by one or more disulfide bonds or associated by other means. The term also includes amino acid polymers in which one or more amino acids are chemical analogs of a corresponding naturally-occurring amino acid and polymers.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the IgA antibody disclosed herein to treat SARS-CoV-2 infection, a therapeutically effective amount is the dosage or concentration of the monoclonal antibody or antigen-binding fragment thereof capable of ameliorating any symptom or marker associated with the infection, preventing or delaying the infection, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

### II. IgA Neutralizing SARS-CoV-2

Widespread reinfections and vaccine breakthrough infections (BTIs) with Omicron have been reported worldwide, and most of the clinically available antibodies have been found to be ineffective against this variant^{5,10,11}. Although Omicron causes less severe symptoms than previous variants, it still results in a substantial number of hospitalizations and deaths, especially in unvaccinated individuals. Antibody pre-exposure therapy could be beneficial for protection of individuals at high risk of developing severe diseases such as immunocompromised patients and elderly individuals, especially in areas/countries with low vaccination/booster rates¹²⁻¹⁵. Recent evidence suggests a shift in the tropism of the Omicron variant towards the upper respiratory tract¹⁶. Viral particles in the upper airways might be more easily released from the nose and mouth, contributing to the increased transmissibility of the Omicron variant¹⁷. The virus might be contained in the upper respiratory tract of those individuals who develop a strong local mucosal immune response, resulting in a mild/asymptomatic infection¹⁸. Thus, mucosal immunity may potentially be exploited for therapeutic or prophylactic purposes¹⁹.

Secretory IgA (sIgA) is the most abundant immunoglobulin type in secretions and is fundamental for mucosal defenses and protection against respiratory viral infections. While serum IgA is predominantly present as a monomer (mIgA), sIgA is composed of two IgA monomers, connected via the joining (J) chain, and associated with the secretory component (SC)²⁰. Dimeric IgA (dIgA) produced by B cells in the mucosa is translocated across the epithelium via the polymeric immunoglobulin receptor (pIgR)²¹. On the luminal side of the epithelium, pIgR is cleaved, while a portion, the SC, remains attached, forming sIgA²². Among the two subclasses of IgA antibodies in human, IgA1 constitutes a higher proportion in the upper respiratory tract (approximately 90%) and IgA2 is more abundant in the lower gastrointestinal tract²²⁻²⁴. Mucosal IgA dominates the neutralizing antibody response to SARS-CoV-2 in the early phase of infection and is more effective in neutralizing SARS-CoV-2^{25,26}, and dIgA has been found to be a more potent neutralizer than IgG or IgA monomers against authentic SARS-CoV-2²⁷⁻²⁹. Thus, delivery of both dIgA and sIgA via nasal spray is potentially the best and convenient option to block viral infection and transmission.

The present disclosure in one aspect provides an immunoglobulin A (IgA) specifically binding to SARS-CoV-2 Omicron variant and neutralizing SARS-CoV-2 Omicron variant.

Binding affinity of the IgA provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g., K_{D}) can be appropriately determined using suitable methods known in the art, including, for example, bio-layer interferometry.

Binding of the IgA to SARS-CoV-2 can also be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assays.

### Specific Anti-SARS-CoV-2 IgA

In some embodiments, the IgA provided herein comprises a paired heavy chain and light chain variable domains derived from the antibody disclosed in the references listed in Table 1.

TABLE 1
Patent Applications Disclosing Anti-SARS-CoV-2 Antibodies

| Publication | |
|---|---|
| Number | Assignee/Applicant |
| WO2021158521A1 | VIR BIOTECHNOLOGY INC., US |
| WO2021169932A1 | INSTITUTE OF MICROBIOLOGY CHINESE ACADEMY OF SCIENCES, CN |
| WO2021173753A1 | VIR BIOTECHNOLOGY INC., US |
| WO2021180602A1 | HARBOUR ANTIBODIES BV, NL et al. |
| WO2021183947A1 | UNIVERSITY OF PITTSBURGH, US |
| WO2021207948A1 | TSB THERAPEUTICS (BEIJING) CO. LTD., CN |
| WO2021203397A1 | TSB THERAPEUTICS (BEIJING) CO. LTD., CN |
| WO2021194188A1 | CELLTRION INC., KR et al. |
| WO2021194985A1 | CENTIVAX INC., US |
| WO2021194886A1 | CENTIVAX INC., US |
| WO2021194896A1 | CENTIVAX INC., US |
| WO2021194965A1 | CENTIVAX INC., US |
| WO2021194951A1 | CENTIVAX INC., US |
| WO2021194891A1 | CENTIVAX INC., dUS |
| WO2021195418A1 | VANDERBILT UNIVERSITY, US |
| WO2021203053A1 | VIR BIOTECHNOLOGY INC., US \| UNIVERSITY OF WASHINGTON, US |
| WO2021211775A1 | VIR BIOTECHNOLOGY INC., US \| HUMABS BIOMED SA, CH |
| WO2021207962A1 | ACTIVE MOTIF SHANGHAI LIMITED, CN |
| WO2021212049A2 | WASHINGTON UNIVERSITY, US |
| WO2021211963A1 | WASHINGTON UNIVERSITY, US |
| WO2021212785A1 | MABWELL (SHANGHAI) BIOSCIENCE CO. LTD., CN |
| WO2021222315A2 | TWIST BIOSCIENCE CORPORATION, US |
| WO2021222316A2 | TWIST BIOSCIENCE CORPORATION, US |
| WO2021222128A1 | FRED HUTCHINSON CANCER RESEARCH CENTER, US |
| WO2021222935A2 | THE ROCKEFELLER UNIVERSITY, US |
| WO2021218947A1 | PEKING UNIVERSITY, CN |
| WO2021226405A1 | INTERNATIONAL AIDS VACCINE INITIATIVE INC., US \| THE SCRIPPS |
| | RESEARCH INSTITUTE, US |
| WO2021228904A1 | ACADEMISCH MEDISCH CENTRUM, NL |
| WO2021231803A2 | ABCORE INC., US \| BARRACLOUGH David, US \| JOHNSON Jennifer, US \| |
| | DWYER Mary, US |
| WO2021229561A1 | THE ISRAEL INSTITUTE OF BIOLOGICAL RESEARCH (IIBR), IL |
| WO2021233834A1 | ASTRAZENECA UK LIMITED, GB |
| WO2021236509A1 | ELPIS BIOPHARMACEUTICALS, US |
| WO2021237051A1 | JECHO LABORATORIES INC., US |
| WO2021237516A1 | SHANGHAI JEMINCARE PHARM CO. LTD., CN \| JIANGXI JEMINCARE |
| | GROUP CO. LTD., CN |
| WO2021238854A1 | NANJING GENSCRIPT BIOTECH CO. LTD., CN |
| WO2021233433A1 | NANJING GENSCRIPT BIOTECH CO. LTD., CN |
| WO2021239935A1 | UNIVERSITÄT ZU KÖLN, DE \| PHILIPPS-UNIVERSITÄT MARBURG, DE |
| WO2021245184A1 | NEURIMMUNE AG, CH |
| WO2021247925A1 | VIR BIOTECHNOLOGY INC., US \| UNIVERSITY OF WASHINGTON, US \| |
| | HUMABS BIOMED SA, CH \| CORTI Davide, CH \| SNELL Gyorgy, US \| |
| | CZUDNOCHOWSKI Nadine, US \| VEESLER David, US \| WALLS Alexandra |
| | C., US \| PARK Young-Jun, US \| TORTORICI M. Alejandra, US |
| WO2021247779A1 | REGENERON PHARMACEUTICALS INC., US |
| WO2021248279A1 | ACTIVE MOTIF SHANGHAI LIMITED, CN \| FUDAN UNIVERSITY, CN \| |
| | SHANGHAI PUBLIC HEALTH CLINICAL CENTER, CN |
| WO2021249547A1 | BIOCYTOGEN PHARMACEUTICALS (BEIJING) CO. LTD., CN |
| WO2021260532A1 | SEOUL NATIONAL UNIVERSITY R&DB FOUNDATION, KR |
| WO2022003530A1 | FONDAZIONE TOSCANA LIFE SCIENCES, IT |
| WO2022026807A2 | ALBERT EINSTEIN COLLEGE OF MEDICINE, US \| MALONIS Ryan, US \| |
| | GEORGIEV George, US \| TONG Karen, US \| LAI Jonathan R., US \| CHANDRAN |
| | Kartik, US |
| WO2022023292A9 | FRIEDRICH-ALEXANDER-UNIVERSITÄT ERLANGEN-NÜRNBERG, DE |
| WO2022037033A1 | CHONGQING MEDICAL UNIVERSITY, CN \| FENG Yulin, CN |
| WO2022036788A1 | CHONGQING MEDICAL UNIVERSITY, CN \| FENG Yulin, CN |
| WO2022046888A1 | TARGET DISCOVERY MERGER SUB II LLC, US |
| WO2022048624A1 | NANJING GENSCRIPT BIOTECH CO. LTD., CN |
| WO2022056171A1 | BIGHAT BIOSCIENCES INC., US |
| WO2022068844A1 | VAZYME BIOTECH CO. LTD., CN |
| WO2022058618A2 | IONTAS LIMITED, GB |
| WO2022062803A1 | THE UNIVERSITY OF HONG KONG, CN |
| WO2022079606A1 | ICOSAGEN CELL FACTORY OÜ, EE |
| WO2022087393A1 | ICAHN SCHOOL OF MEDICINE AT MOUNT SINAI, US |
| WO2022094622A1 | THE UNIVERSITY OF CHICAGO, US |
| WO2022098870A1 | THE ROCKEFELLER UNIVERSITY, US |
| WO2022095968A1 | SHANGHAITECH UNIVERSITY, CN \| SHOX SCIENCE LIMITED, CN |
| WO2022099187A2 | PRESIDENT AND FELLOWS OF HARVARD COLLEGE, US |
| WO2022103245A1 | SHAPERON INC., KR \| HUVETBIO INC., KR \| SEOUL NATIONAL |
| | UNIVERSITY R&DB FOUNDATION, KR |
| WO2022115486A1 | VIR BIOTECHNOLOGY INC., US \| HUMABS BIOMED SA, CH |
| WO2022114426A1 | OSONG MEDICAL INNOVATION FOUNDATION, KR |
| WO2022120375A1 | THE UNIVERSITY OF CHICAGO, US \| WISCONSIN ALUMNI RESEARCH |
| | FOUNDATION, US |
| WO2022132887A1 | BOARD OF REGENTS THE UNIVERSITY OF TEXAS SYSTEM, US \| |
| | GOLLIHAR Jimmy Dale, US |
| WO2022140422A1 | CONSEJO NACIONAL DE INVESTIGACIONES CIENTÍFICAS Y TÉCNICAS |
| | (CONICET), AR \| INSTITUTO NACIONAL DE TECNOLOGÍA |
| | AGROPECUARIA, AR \| VIRGINIA POLYTECHNIC INSTITUTE AND STATE |
| | UNIVERSITY, US |
| WO2022133545A1 | GARVAN INSTITUTE OF MEDICAL RESEARCH, AU |
| WO2022150654A2 | 10X GENOMICS INC., US |
| WO2022150660A1 | 10X GENOMICS INC., US |
| WO2022153212A1 | AXON NEUROSCIENCE SE, CY |
| WO2022155324A1 | THE ROCKEFELLER UNIVERSITY, US |
| WO2022158497A1 | MABPROTEIN CO. LTD., JP |
| WO2022159839A1 | THE HENRY M. JACKSON FOUNDATION FOR THE ADVANCEMENT OF |
| | MILITARY MEDICINE INC., US \| WALTER REED ARMY INSTITUTE OF |
| | RESEARCH, US |
| WO2022161491A1 | INSTITUT PASTEUR OF SHANGHAI CHINESE ACADEMY OF |
| | SCIENCES, CN \| NANJING ADVANCED ACADEMY OF LIFE AND |
| | HEALTH, CN |
| WO2022167815A1 | RQ BIOTECHNOLOGY LIMITED, GB |
| WO2022167816A2 | OXFORD UNIVERSITY INNOVATION LIMITED, GB |
| WO2022170126A2 | ADAGIO THERAPEUTICS INC., US |
| WO2022173670A1 | THE UNITED STATES OF AMERICA AS REPRESENTED BY THE |
| | SECRETARY DEPARTMENT OF HEALTH AND HUMAN SERVICES, US |
| WO2022179561A1 | THE UNIVERSITY OF HONG KONG, CN \| CENTRE FOR VIROLOGY |
| | VACCINOLOGY AND THERAPEUTICS LIMITED, CN |
| WO2022188829A1 | SHANGHAI JUNSHI BIOSCIENCES CO. LTD., CN \| SUZHOU JUNMENG |
| | BIOSCIENCES CO. LTD., CN |
| WO2022192661A1 | BOARD OF REGENTS THE UNIVERSITY OF TEXAS SYSTEM, US \| |
| | GOLLIHAR Jimmy Dale, US |
| WO2022204202A1 | VIR BIOTECHNOLOGY INC., US |
| WO2022208453A1 | SIDHU Sachdev, CA \| MIERSCH Shane, CA \| CHEN Gang, CA \| LABRIOLA |
| | Jonathan, CA \| THE GOVERNING COUNCIL OF THE UNIVERSITY OF |
| | TORONTO, CA |
| WO2022212910A1 | ADITXT INC., US |
| WO2022221120A1 | BIO-TECHNE CORPORATION, US \| RESNIK Ernesto Rubin, US \| BAJER Anna |
| | Agnieszka, US \| MUNSHI Cyrus B., US \| BOSTROM Kerri Ann, US \| |
| | BONNEVIER Jody, US \| GOETZ Christine Andrea, US |
| WO2022221314A1 | BIOLEGEND INC., US |
| WO2022226079A1 | INBIOS INTERNATIONAL INC., US |
| WO2022228827A1 | INSTITUT PASTEUR, FR |
| WO2022231320A1 | KOOKMIN UNIVERSITY INDUSTRY ACADEMY COOPERATION |
| | FOUNDATION, KR |
| WO2022231321A1 | KOOKMIN UNIVERSITY INDUSTRY ACADEMY COOPERATION |
| | FOUNDATION, KR |
| WO2022235867A2 | THE ROCKEFELLER UNIVERSITY, US |
| WO2022237924A1 | USTAV ORGANICKE CHEMIE A BIOCHEMIE AV CR V. V. I., CZ \| |
| | BIOTECHNOLOGICKY USTAV AV CR V. V. I., CZ |
| WO2022245288A1 | NATIONAL UNIVERSITY OF SINGAPORE, SG |
| WO2022251403A1 | ICAHN SCHOOL OF MEDICINE AT MOUNT SINAI, US |

In some embodiments, the IgA disclosed herein comprises a paired heavy chain and light chain variable domains of an antibody selected from the group consisting of casirivimab (Regeneron Pharmaceuticals), imdevimab (Regeneron Pharmaceuticals), etesevimab (Eh Lilly and Company), bamlanivimab (Eh Lilly and Company), CT-P59 (Celltrion Healthcare), BRII-196 (Brii Biosciences), BRII-198 (Brii Biosciences), VIR-7831 (Vir Biotechnology), AZD7442 (AstraZeneca), AZD8895 (AstraZeneca), AZD1061 (AstraZeneca), TY027 (Tychan Pte. Ltd.), SCTA01 (Sinocelltech Ltd.), MW33 (Mabwell Bioscience Co., Ltd.), JS016 (Junshi Biosciences), DXP593 (Singlomics/Beigene), DXP604 (Singlomics/Beigene), STI-2020 (Sorrento Therapeutics), BI 767551/DZIF-lOc (U. Cologne/Boehringer Ingelheim), COR-101 (CORAT Therapeutics), HLX70 (Hengenix Biotech), ADM03820 (Ology Bioservices), HFB30132A (HiFiBiO Therapeutics), ABBV-47D11 (AbbVie), C144-LS (Bristol-Myers Squibb, Rockefeller University), C-135-LS (Bristol-Myers Squibb, Rockefeller University), LY-CovMab (Luye Pharma), JMB2002 (Jemincare), ADG20 (Adagio Therapeutics), LY-Cov1404 (AbCellera; Eh Lilly and Company).

In some embodiments, the IgA antibody disclosed herein is DXP-604 IgA antibodies which comprises a heavy chain comprises or consists of the amino acid sequence set forth in SEQ ID NO: 1, and a light chain comprises or consists of the amino acid sequence set forth in SEQ ID NO: 2; or variants thereof wherein the heavy chain and/or light chain has one, two, or three amino acid substitutions, additions, deletions or combination thereof.

In some embodiments, the IgA antibody disclosed herein is dimeric. In some embodiments, the IgA antibody disclosed herein further comprises a J chain comprises or consists of the amino acid sequence set forth in SEQ ID NO: 3, or a sequence variant thereof comprising one, two, or three acid substitutions, additions, deletions or combination thereof.

In some embodiments, the IgA antibody disclosed herein is secretory. In some embodiments, the IgA antibody disclosed herein further comprises a secretory component comprises or consists of the amino acid sequence set forth in SEQ ID NO: 4, or a sequence variant thereof comprising one, two, or three acid substitutions, additions, deletions or combination thereof.

TABLE 2
Amino acid sequences of recombinant DXP-604 IgA antibodies

| | SEQ | |
|---|---|---|
| Description | ID NO | Amino Acid Sequence |
| Heavy chain | 1 | EVQLVESGGGLIQPGGSLRLSCAASGIIVSSNYMTWV |
| | | RQAPGKGLEWVSVIYSGGSTFYADSVKGRFTISRDNS |
| | | KNTLYLQMSSLRAEDTAVYYCARDLGPYGMDVWGQ |
| | | GTTVTVSSASPTSPKVFPLSLCSTQPDGNVVIACLVQG |
| | | FFPQEPLSVTWSESGQGVTARNFPPSQDASGDLYTTSS |
| | | QLTLPATQCLAGKSVTCHVKHYTNPSQDVTVPCPVPS |
| | | TPPTPSPSTPPTPSPSCCHPRLSLHRPALEDLLLGSEAN |
| | | LTCTLTGLRDASGVTFTWTPSSGKSAVQGPPERDLCG |
| | | CYSVSSVLPGCAEPWNHGKTFTCTAAYPESKTPLTAT |
| | | LSKSGNTFRPEVHLLPPPSEELALNELVTLTCLARGFSP |
| | | KDVLVRWLQGSQELPREKYLTWASRQEPSQGTTTFA |
| | | VTSILRVAAEDWKKGDTFSCMVGHEALPLAFTQKTID |
| | | RLAGKPTHVNVSVVMAEVDGTCY |
| Light chain | 2 | DIQLTQSPSFLSASVGDRVTITCRASQGISSDLAWYQQ |
| | | KPGKAPNLLIYAASTLQSGVPSRFSGSGSGTEFTLTISS |
| | | LQPEDFATYYCQQLNSDLYTFGQGTKLEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| J chain | 3 | QEDERIVLVDNKCKCARITSRIIRSSEDPNEDIVERNIRI |
| | | IVPLNNRENISDPTSPLRTRFVYHLSDLCKKCDPTEVE |
| | | LDNQIVTATQSNICDEDSATETCYTYDRNKCYTAVVP |
| | | LVYGGETKMVETALTPDACYPD |
| Secretory | 4 | KSPIFGPEEVNSVEGNSVSITCYYPPTSVNRHTRKYWC |
| component | | RQGARGGCITLISSEGYVSSKYAGRANLTNFPENGTFV |
| | | VNIAQLSQDDSGRYKCGLGINSRGLSFDVSLEVSQGP |
| | | GLLNDTKVYTVDLGRTVTINCPFKTENAQKRKSLYK |
| | | QIGLYPVLVIDSSGYVNPNYTGRIRLDIQGTGQLLFSV |
| | | VINQLRLSDAGQYLCQAGDDSNSNKKNADLQVLKPE |
| | | PELVYEDLRGSVTFHCALGPEVANVAKFLCRQSSGEN |
| | | CDVVVNTLGKRAPAFEGRILLNPQDKDGSFSVVITGL |
| | | RKEDAGRYLCGAHSDGQLQEGSPIQAWQLFVNEESTI |
| | | PRSPTVVKGVAGGSVAVLCPYNRKESKSIKYWCLWE |
| | | GAQNGRCPLLVDSEGWVKAQYEGRLSLLEEPGNGTF |
| | | TVILNQLTSRDAGFYWCLTNGDTLWRTTVEIKIIEGEP |
| | | NLKVPGNVTAVLGETLKVPCHFPCKFSSYEKYWCKW |
| | | NNTGCQALPSQDEGPSKAFVNCDENSRLVSLTLNLVT |
| | | RADEGWYWCGVKQGHFYGETAAVYVAVEERKAAG |
| | | SRDVSLAKADAAPDEKVLDSGFREIENKAIQDPR |

In certain embodiments, the IgA provided herein are humanized. A humanized IgA is desirable in its reduced immunogenicity in human. A humanized IgA is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al., Nature (1986) 321:522-525; Riechmann et al., Nature (1988) 332:323-327; Verhoeyen et al., Science (1988) 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., J. Immunol. (1993) 151:2296; Chothia et al., J. Mot. Biol. (1987) 196:901). Alternatively, a framework derived from the consensus sequence of all human antibodies may be used for the grafting of the non-human CDRs (see, for example, Carter et at. Proc. Natl. Acad. Sci. USA (1992) 89:4285; Presta et al., J. Immunol. (1993) 151:2623).

In certain embodiments, the humanized IgA provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody.

### Polynucleotides and Recombinant Methods

The present disclosure provides isolated polynucleotides that encode the IgA disclosed herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-SARS-CoV-2 IgA disclosed herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

The present disclosure provides vectors (e.g., expression vectors) containing the nucleic acid sequence provided herein encoding the anti-SARS-CoV-2 IgA, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the IgA disclosed herein can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-SARS-CoV-2 IgA-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of the IgA provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. (1977) 36:59); baby hamster kidney cells (BHK, ATCC CCL 10); Chinese Hamster Ovary cells (CHO), CHO cells deficient in dihydrofolate reductase (DHFR) activity, CHO-DHFR (Urlaub et al., Proc. Natl. Acad. Sci. USA (1980) 77:4216); mouse sertoli cells (TM4, Mather, Biol. Reprod. (1980) 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. (1982) 383:44-68); MRC 5 cells; FS4 cells; and a human hepatoma line (Hep G2). In some preferable embodiments, the host cell is 293F cell.

Host cells are transformed with the above-described expression or cloning vectors for IgA production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the IgA provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. (1980) 102:255, U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the IgA can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology (1992) 10:163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-SARS-CoV-2 IgA prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Purification

In certain embodiments, the IgA of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an IgA of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Pharmaceutical Composition

The present disclosure further provides pharmaceutical compositions comprising the anti-SARS-CoV-2 IgA disclosed herein and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agents. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CCR4 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

In certain embodiments, the pharmaceutical compositions comprising the anti-SARS-CoV-2 IgA described herein further comprise one or more additional therapeutic agents that are co-administered with the IgA. It can be understood that the additional therapeutic agents can be co-formulated with the IgA, or be mixed with the IgA right before the administration, such as in the IV infusion bag.

### IV. Methods of Use of IgA

The present disclosure also provides therapeutic methods comprising: administering a therapeutically effective amount of the IgA as provided herein to a subject in need thereof, thereby treating the infection of SARS-CoV-2.

The therapeutically effective amount of an IgA as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the IgA as provided herein may be administered at a therapeutically effective dosage of about 0.0001 mg/kg to about 100 mg/kg. In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The IgA disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes. In some preferred embodiments, the IgA disclosed herein may be administered via intranasal route.

In some embodiments, the IgA disclosed herein may be administered alone or in combination with one or more additional therapeutic means or agents. For example, the IgA disclosed herein may be administered in combination with another therapeutic agent.

In certain of these embodiments, an IgA as disclosed herein that is administered in combination with one or more additional therapeutic agents may be administered simultaneously with the one or more additional therapeutic agents, and in certain of these embodiments the IgA and the additional therapeutic agent(s) may be administered as part of the same pharmaceutical composition. However, an IgA administered “in combination” with another therapeutic agent does not have to be administered simultaneously with or in the same composition as the agent. An IgA administered prior to or after another agent is considered to be administered “in combination” with that agent as the phrase is used herein, even if the IgA and second agent are administered via different routes. Where possible, additional therapeutic agents administered in combination with the IgA disclosed herein are administered according to the schedule listed in the product information sheet of the additional therapeutic agent, or according to the Prescriber's Digital Reference (available online only at pdr.net) or protocols well known in the art.

The dose of the agent for the combination therapy can be determined by the existence, nature and extent of any adverse side effects that might accompany the administration of a particular agent. Typically, the attending physician will decide the dosage of the agent for the combination therapy with which to treat each individual patient, taking into consideration a variety of factors, such as age, body weight, general health, diet, sex, the agent be administered, route of administration, and the severity of the condition being treated. By way of example and not intending to limit the present disclosure, the dose for the combination therapy can be about 0.0001 to about 1 g/kg body weight of the subject being treated/day, from about 0.0001 to about 0.001 g/kg body weight/day, or about 0.01 mg to about 1 g/kg bodyweight/day. Dosage units may be also expressed in mg/m², which refer to the quantity in milligrams per square meter of body surface area.

Each therapeutic agent in the combination therapy described herein may be administered simultaneously (e.g., in the same medicament or at the same time), concurrently (i.e., in separate medicaments administered one right after the other in any order or sequentially in any order. Sequential administration may be useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In certain embodiments, the IgA of the present disclosure and the second drug are combined or co-formulated in a single dosage form. In certain embodiments, the IgA of the present disclosure and the second drug are administered separately. Although the simultaneous administration of the IgA of the present disclosure and the second drug may be maintained throughout a period of treatment, anti-cancer activity may also be achieved by subsequent administration of one compound in isolation (for example, the IgA following initial combination treatment, or alternatively, the second drug following initial combination treatment). In some embodiments, the IgA is administered before administration of the second drug, while in other embodiments, the IgA is administered after administration of the second drug. In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

### EXAMPLES

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

Secretory IgA Anti-RBD Antibodies are Produced at a Lower Level Following Vaccination as Compared to after Breakthrough Infections.

Among immunoglobulins, sIgA are present at highest concentration in saliva and constitute an accessible marker of the mucosal immune response to SARS-CoV-2^{30,31}. We previously showed that in individuals receiving mRNA vaccines, higher levels of salivary anti-RBD sIgA, but not plasma anti-RBD IgG or IgA, or salivary anti-RBD IgG or IgM antibodies, were associated with protection against BT1³². Here we further tested the presence of salivary antibodies against the RBDs of G614 (wild-type) and all Omicron lineages in individuals with different vaccination types and follow the dynamic of salivary IgA and IgG antibody responses over time (up to 9 months)³² (Table 3).

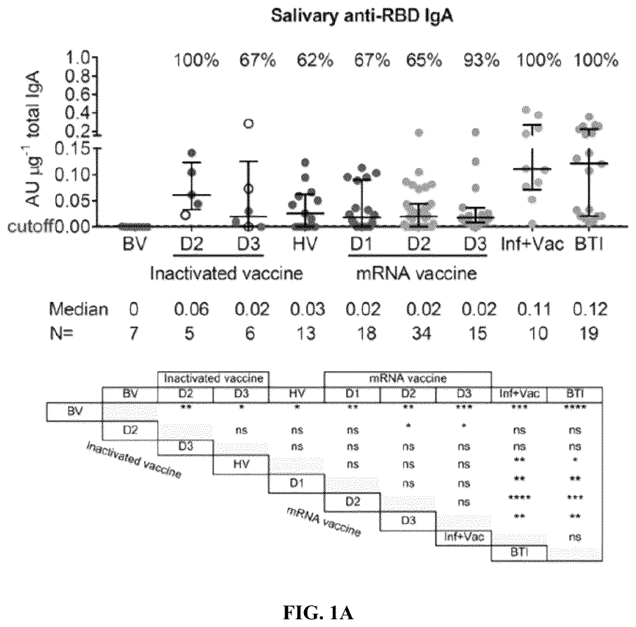

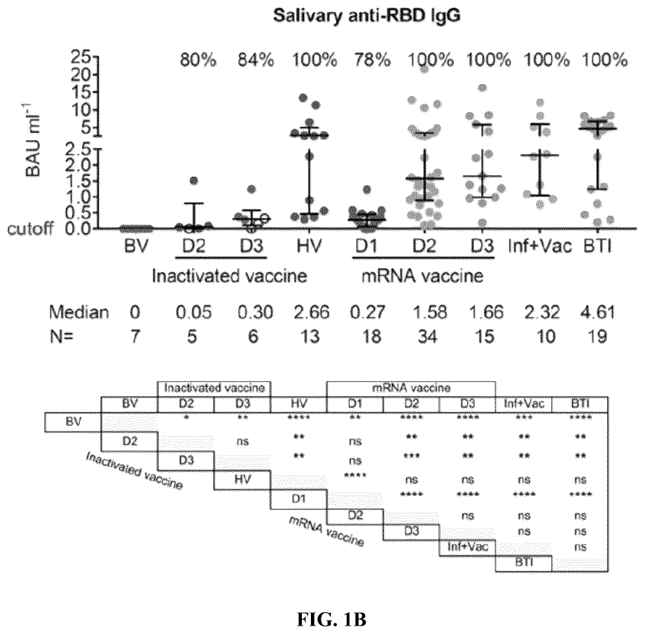

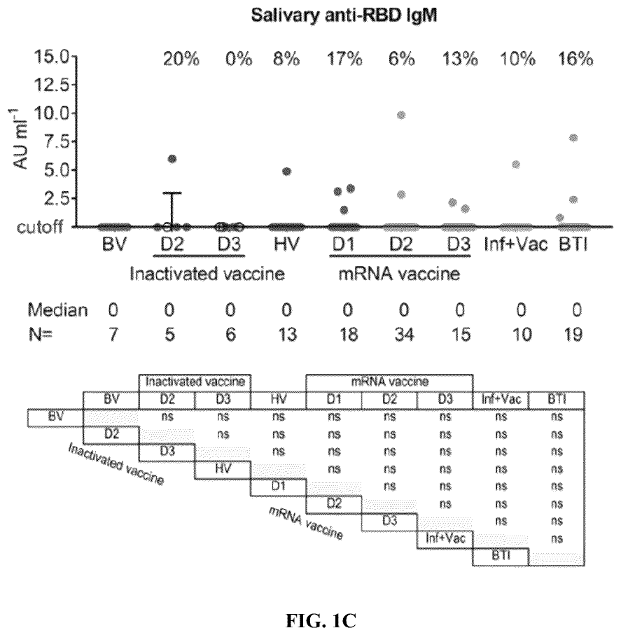

The results show that the median salivary anti-RBD IgA levels in individuals receiving two or three doses of inactivated whole-virion SARS-CoV-2, one to three doses mRNA vaccine or heterologous vaccination were lower than that after BTI or a mRNA vaccine booster after infection (FIG. 1A). Salivary anti-RBD IgG levels after the second and third dose of mRNA vaccine or heterologous mRNA booster dose were similar to that measured after BTI (FIG. 1B). Furthermore, salivary IgM anti-RBD antibodies were detected in less than 20% of individuals after vaccination and BTI (FIG. 1C). Levels of salivary anti-RBD IgA antibodies correlated better with levels of RBD-specific secretory immunoglobulin (sIg) (R=0.789, P<0.0001) than plasma IgA antibodies (R=0.256, P<0.0046), suggesting that most of the salivary IgA antibodies measured were produced locally in the salivary glands as sIgA rather than derived from the plasma³². On the contrast, salivary anti-RBD IgG antibodies correlated with plasma IgG antibodies, implying that those antibodies were mainly derived from plasma (R=0.704, P<0.0001).

Salivary RBD-specific IgA antibodies were produced at a low level after the third mRNA vaccine dose and gradually decreased but were still detectable in nearly 85% individuals up to 9 months, while after BTI, these antibodies were sustained at a higher level. Lower salivary IgA (from to 2.1- to >2.8-fold) and IgG (from 2.4- to 6.1-fold) antibody levels against the RBDs of BA.1, BA.2 and BA.4/5 compared to G614 RBD were observed in vaccinated individuals within two months after two or three doses of mRNA vaccine, but no decrease in antibody levels against Omicron variants was observed in individuals that experienced BTIs during the Omicron BA.1 wave. Furthermore, a significant increase (by more than 10-fold) of salivary IgA and IgG antibodies against Omicron subvariants RBD was observed 2-6 weeks after BTI. In conclusion, both BTI and vaccination can boost the viral specific IgG response; however, BTI, unlike vaccination, can also boost the mucosal sIgA response against SARS-CoV-2³³, most likely due to local stimulation of nasal associated lymphoid tissues.

The low or sufficient level of sIgA antibodies induced by the intramuscular mRNA and/or inactivated vaccines, particularly against various Omicron lineages, suggest a low primary and booster mucosal immune response. Thus, development of vaccination strategies that can improve the mucosal IgA response, together with an effective monoclonal antibody therapy that can be delivered directly at the mucosal surface, are priorities in the current stage of the pandemic.

### Example 2

### Characterization of Parental Neutralizing IgG Antibodies

Four neutralizing IgG mAbs, 01A05 (isolated in this study), rmAb23³⁴, DXP-604^{2,35}, and XG014^{36,37}, targeting SARS-CoV-2 RBD, were used for conversion into IgA formats. All IgG mAbs were isolated from convalescent patients who had the wild-type strain (the Wuhan or G614 strain) infection before the emergence of Omicron³⁴⁻³⁷.

The antibodies were initially compared for their ability to bind to different RBD epitopes. Neutralizing anti-SARS-CoV-2 antibodies can be categorized into four³⁸ or six³⁹ classes based on their mode of binding to the S protein. Computational structure modelling was performed based on binding of 01A05 to RBD of variants (Alpha, Beta, Delta, and Omicron) or through previously published information (Protein Data Bank (PDB) files) describing docking (rmAb23)³⁴ or crystallization (DXP-604 and XG014) studies^{35,36}. 01A05, encoded by IGHV1-18 and IGKV1-39, is a class I antibody with a long CDR3 (27 amino acids, aa) that mainly binds the part of a receptor-binding motif that is accessible when the RBD is in the up conformation. rmAb23 and DXP-604 are both IGHV3-53- and IGKV1-9-encoded antibodies with a short CDR3 (11 aa)³⁴, characteristic of class I antibodies that bind to RBD in the up conformation. XG014 (IGHV5-51/IGLV1-51) is a class IV antibody that recognizes a conserved epitope outside the receptor-binding motif (RBM) in the RBD and locks all three RBDs in the S-trimer in the down conformation, preventing binding to ACE2³⁶. Three 01A05 antibodies can simultaneously bind three RBDs of one S-trimer in the up position (3-RBD up), but only one rmAb23 or DXP-604 Fab can bind the trimeric S protein (3-RBD up) due to steric hindrance caused by the other Fabs. Three XG014 Fabs can bind all three RBD in the down conformation (3-RBD down) and XG014 should also be able to bind RBD in the up position according to structural simulations. In summary, the four antibodies recognize different epitopes in the RBD, with the Fabs of 01A05 and rmAb23 binding G614 RBD with lower affinity (a dissociation constant (KD) of 2.5 nM and 6.5 nM³⁴, respectively) compared to those of DXP-604 and XG014 that show subnanomolar KD^{2,35-37}.

The four IgG mAbs were compared for binding to RBDs derived from G614, Alpha, Beta, Delta and Omicron (BA.1, BA.2 and BA.4/5) by ELISA and for neutralization of these variants by microneutralization assay with authentic viruses. All four antibodies bound RBDs from G614, Alpha and Delta with a one-half maximal effective concentration (EC₅₀) value from 0.01 to 1.89 nM but only DXP-604 and XG014 bound to RBDs from Beta, and Omicron BA. 1, BA.2, BA.4/5 (EC50 from 0.33 to 9.48 nM). The antibodies 01A05, rmAb23, DXP-604 and XG014 neutralized authentic G614 SARS-CoV-2 virus at half maximal inhibitory concentration (IC₅₀) values of 0.18, 4.44, 0.3 and 0.24 nM, respectively. In accordance with the ELISA results, 01A5 neutralized only the VOCs Alpha and Delta (IC₅₀: 10.41 and 8.62 nM, respectively), rmAb23 neutralized only Delta (IC₅₀: 14.45 nM) while XG014 efficiently neutralized Alpha, Beta and Delta (IC₅₀ values from 0.79 to 2.34 nM). Only DXP-604 neutralized all VOCs although it was less effective against Omicron BA.1 (IC₅₀: 22.93 nM) and BA.5 (IC₅₀: 12.36 nM) than the other variants (IC₅₀ values from 2.63 to 7.71 nM).

The binding and neutralization assay results are in accordance with structural models showing that certain mutations in the RBDs of VOCs are located in the binding epitope of 01A05 and rmAb23 and may therefore affect the binding and neutralization of Alpha (N501Y), Delta (T478K), Beta (N501Y, E484K) and Omicron (including K417N, G446S, E484K, F486V, Q493R, G496S, N501Y). However, mutations in RBD residues within the binding epitope of DXP-604, such as N501Y and T478K, resulted in a less pronounced decrease in neutralization activity against Alpha, Beta, and Delta. Interestingly, DXP-604 maintained neutralizing activity against the Omicron BA.1, BA.2 and BA.4/5, even though the RBD harboured more than 6 substitutions in the mAb epitope, including K417N, S477N, T478K, Q498R, N501Y, and Y505H. The epitope recognized by XG014 is mostly outside the hotspots of the RBM, where prevalent mutations are located, explaining the high and broad neutralization potency against Alpha, Beta, and Delta⁴⁰. Therefore, among the four antibodies analyzed, DXP-604 appeared to be the most broadly effective neutralizer, even when a few residues in its binding epitope are substituted.

The apparent resistance of DXP-604 to SARS-CoV-2 mutations was confirmed in a S-pseudotype vesicular stomatitis virus (VSV) neutralization assay showing it potent neutralizing effect against 15 known SARS-CoV-2 variants (IC₅₀ from 0.03 nM to 1.6 nM), including D614G, Alpha (B.1.1.7), Beta 453 (B.1.351), Gamma (P.1), Delta (B.1.617.2), 454 Kappa (B.1.617.1), Delta plus (AY.1), Mu (B.1.621), Lambda (C.37) and Iota (B.1.526), BA.1, BA.2, BA.3, BA.1/BA.2 subvariants BA.1.1 (BA. 1+R346K), BA.2.12.1 (BA.1+L452Q+S704L), BA.2. 13 (BA.1+L452M), and other clade 1b sarbecoviruses circulating among other species, including RaTG13 (IC₅₀: 0.01 nM) and Pangolin-GD⁴¹ (IC₅₀: 0.05 nM). Although the binding of most class I neutralizing antibodies is completely abolished by the K417N and E484K/A mutations in Beta and Omicron, DXP-604 exhibited potent neutralization activity against variants carrying these mutations^{37,42}. These data suggest that, despite reduced efficacy against Omicron VOC², DXP-604 is a broadly effective neutralizing antibody against clade 1b sarbecoviruses, in contrast to most other class I antibodies²⁴³.

DXP-604 recognizes an epitope that almost completely overlaps the binding site of ACE2³⁵ and one single DXP-604 Fab bound to the S-trimer with 3-RBD in the up position can prevent binding of ACE2 to all three S monomers. Further analysis of the X-ray crystal structure³⁵ revealed that the footprint of the DXP-604 heavy chain on the RBD is similar to that of LY-CoVO16 (etesevimab); however, compared to the footprint of the LY-CoV016 light chain on the RBD, a higher degree of overlap exists between that of the DXP-604 light chain and ACE2-contact surface. Hydrogen bonds were formed between S30/S67 in the light chain of DXP-604 and RBD Q498, which is a key ACE2-binding site⁴⁴, and between the main chain groups of Q27/S28 and RBD G502. These characteristics were not observed for LY-CoVO16 and RBD interactions and may account for the superior, broadly neutralizing capacity of DXP-604 compared to other class I antibodies². Thus, in contrast to 01A05, rmAb23 and other IGHV3-53/3-66 public antibodies, the high binding affinity and ACE2-mimicking epitope enables DXP-604 to exhibit a higher tolerance for RBD substitutions and to retain a broad neutralization activity.

### Example 3

Dimerization of IgA1 Antibodies Increases the Neutralization Potency of DXP-604, 01A05, rmAb23 and XG014

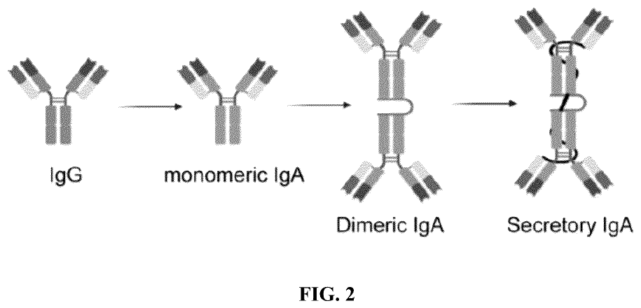

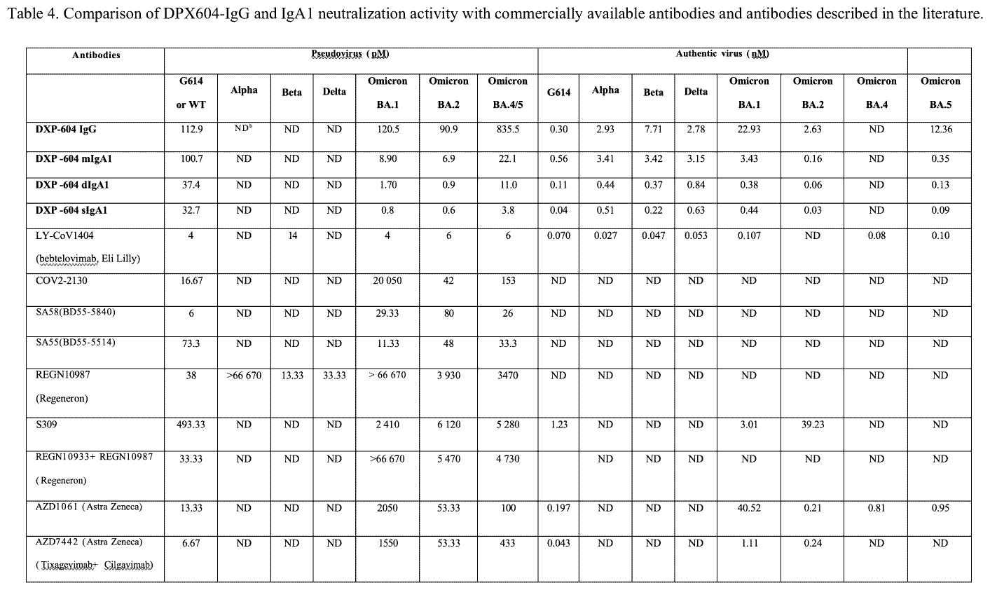

In mucosal tissues, IgA is mainly produced as a dimer of two IgA monomers covalently linked together via a J chain and associated with the SC. As sIgA1 is the main immunoglobulin type in the upper respiratory tract, four antibodies were engineered as monomeric (mIgA1), dimeric (dIgA1, via co-expression of the J chain), and secretory (sIgA1, by co-expression of the J chain and SC) IgA1 antibodies to compare the binding and neutralizing properties of the various forms of IgA1 (FIG. 2).

The conversion of IgG to IgA1 did not strongly increase the binding affinity of the antibodies for the RBD in ELISA (less than two-fold change) with the exception of an increase in DXP-604 mIgA1 binding to the BA.2 (EC₅₀: 1.42 nM) and rmAb23 binding to the Alpha RBD (EC₅₀: 0.35 nM), which were 6.7 and 5.4-fold higher than that of the parental IgG antibodies (EC₅₀: 9.48 and 1.89 nM, respectively). However, dimerization of IgA1 greatly increased the binding of dimeric and secretory IgA1 DXP-604 antibodies to the RBD of the Omicron BA.1, BA.2 and BA.4/5 (EC₅₀ from 0.08 to 0.51 nM) from 7.3- to 50.4-fold compared to that of the parental IgG antibodies and from 2.8- to 9.9-fold increase compared to that of the corresponding mIgA1 antibodies. In addition, XG014 dimeric and secretory IgA1 bound to the BA.2 and BA.4/5 RBDs (EC₅₀ from 0.07 to 0.13 nM) 4.7- to 9.9-fold more efficiently than the parental IgG antibodies and 2.7- to 4.5-fold more efficiently than the corresponding mIgA1 antibodies.

Switching from IgG to IgA1 and dimerization increased antibody neutralization activity against real virus, and the effect was greatest for DXP-604, 01A05, and rmAb23. DXP-604 monomeric IgA1 showed increased neutralization activity against BA.1 (IC₅₀: 3.43 nM, by 6.7-fold), BA.2 (IC₅₀: 0.16 nM, by 17.0-fold) and BA.5 (IC₅₀: 0.35 nM, by 35.5-fold). More importantly, DXP-604 dimeric and secretory IgA1 showed increased neutralization activity against all variants but particularly against the Omicron lineages BA.1, BA.2 and BA.5, which was 47.8- to 143-fold higher than the parental IgG and 2.7- to 9.0-fold higher than the monomeric IgA1. DXP-604 dimeric and secretory IgA1 neutralized Omicron BA.1 (IC₅₀: 0.38 and 0.44 nM, respectively), BA.2 (IC₅₀: 0.06 and 0.03 nM, respectively) and BA.5 (IC₅₀: 0.13 and 0.09 nM, respectively) to a level similar to the neutralization of G614 (IC₅₀: 0.11 and 0.04 nM, respectively) and to the counterpart IgG antibodies against G614 (IC₅₀: 0.30 nM).

An increase in neutralization activity by mIgA1 compared to parental IgG was also observed for 01A05 against G614 (IC₅₀: 0.04 nM, by 4.4-fold) and Alpha (IC₅₀: 0.93 nM, by 11.2-fold), and for rmAb23 against G614 (IC₅₀: 0.89 nM, by 5.0-fold), Alpha (1.66 nM, by >100.6-fold) and Delta (IC₅₀: 3.42 nM, by 4.2-fold). Dimerization also improved the neutralizing activity of 01A05 and rmAb23 against G614, Alpha and Delta by 4.5- to >179-fold compared to IgG and up to 12.0-fold compared to mIgA1. Interestingly, switching to IgA1 and dimerization rescued the neutralizing activity of rmAb23 against Alpha, to some extent (IC₅₀: 1.79 and 0.93 nM, for dIgA1 and sIgA1, respectively) and XG014 against Omicron BA. 1 (IC₅₀: 3.29 and 4.44 nM, for dIgA1 and sIgA1, respectively). These results suggest that the conversion of IgG to IgA1, particularly to the dimeric and secretory forms of IgA1, improved the neutralizing potency of the antibodies against various VOCs.

### Example 4

Increased Neutralizing Potency of Dimeric IgA1 is Associated with Increased Avidity

Enhanced neutralization of dimeric IgA1 antibodies exhibited different patterns, suggesting that the epitope and affinity, in addition to valency, may affect antibody potency⁴⁵. The increase in neutralization potency was more profound against variants for which the parental IgG showed lower (but presence of) neutralization activity. In fact, the fold-change improvement in binding and neutralizing activity of the IgA1 forms was positively correlated with the EC₅₀ values of the parental IgG²⁸. For example, DXP-604, which showed a low RBD binding (EC₅₀: 1.60 nM) and a low neutralizing activity (IC₅₀: 12.36 nM) against BA.5 as an IgG, was found to be 20.5-fold more potent in binding the RBD and 92.0-fold more potent in neutralizing BA.5 as a dIgA1. In addition, for DXP-604, the fold-change increase in RBD binding correlated with the fold-change increase in neutralization activity, suggesting that an increase in neutralizing activity is associated with an increased affinity for the RBD, at least for this antibody for which correlation analysis could be individually performed due to broad neutralization.

As the EC50 value obtained by ELISA does not measure affinity, and since DXP-604 dimeric and secretory IgA1 neutralized Omicron including the circulating BA.5 lineage with a high potency, we further characterized the binding properties of this antibody. The apparent increase in avidity of antibodies could be due to inter- or intra-S cross-linking. According to in silico models, linking of two RBDs on the same S would be feasible for both monomeric or dimeric 01A05 and XG014 antibodies, however only one DXP-604 Fab can bind to each S due to steric hindrance caused by the light chain, thus preventing intra-S binding. We used an avidity assay by surface plasmon resonance (SPR) to experimentally confirm that the dimeric antibody can simultaneously engage two RBDs on different S proteins⁴⁶. The association rate, measured as a control, was not affected by avidity and, indeed, was not affected by antigen concentration. A slower dissociation rate was observed for dimeric IgA1 DXP-604 at higher concentration of immobilized antigen, consistent with inter-molecular avidity effects. At lower concentrations of immobilized antigen, the kd of dIgA1 was faster as intermolecular binding was prevented by the increased distance between RBD molecules, which resulted in loss of avidity. The S-trimers on the surface of SARS-CoV-2 float readily and are widely spaced (a mean of 24 trimeric S protein per virus)⁴⁷ at an average distance of 25 nm⁴⁸. Theoretically, IgG and mIgA1 antibodies may bind RBD on two different S-trimers spaced by ˜2 nm distance. Structural simulations show that dIgA1 and sIgA1 can bridge upon S-trimers ˜11 nm apart. It is plausible that the dimeric forms are more likely to engage in intermolecular binding on the viral surface. Thus, the increased neutralization potency of dIgA1 and sIgA1 appears to be, at least partly, due to increased avidity mediated by inter-S-trimer binding on the viral surface but other mechanisms such as aggregation may also be involved.

### Example 5

### Dimerization of IgA1 Antibodies Increases the Neutralization Potency Against Emerging Omicron Subvariants

According to WHO, more than 300 sublineages of Omicron are circulating globally with the most dominant variants consisting of BA.5 and its descendants, including BQ.1, BQ.1.1, BA.5.2 and BF.7, in addition to BA.2 descendants such as BA.2.75, BA.2.75.2, BR.2, BN.1, CH.1. 1 and XBB. The BQ.1 and BQ.1.1 subvariants are now dominant in parts of Europe and North America, while BF.7 and XBB are dominant in some regions of Asia. In China, where the infections are surged rapidly now, the BF.7 subvariant is dominant in Beijing, whereas BA.5.2 seems to be more prevalent in Guangzhou, two large cities in the Northern and Southern parts of China respectively. Compared to BA.5, BQ.1 carries two additional mutations (K444T and N460K) in RBD while BQ.1.1 carries an additional RBD mutation (R346T). BA.2.75 carry three mutations (G446S, N460K, and rev R493Q) in RBD compared to BA.2 while BA.2.75.2 contains two additional mutations (R346T and F486S). The S protein of XBB has 14 mutations in addition to those found in BA.2, including 9 in the RBD (G339H, R346T, L368I, V445P, G446S, N460K, F486S, F490S and reverse (rev) R493Q), whereas XBB. 1 has an additional G252V mutation. BA.2.75.2, BQ.1, BQ.1.1, XBB and XBB.1 subvariants are resistant to the majority of antibodies approved for use in the clinic, including bebtelovimab (LY-CoV1404) and evusheld (combination of tixagevimab (AZD8895) and cilgavimab (AZD1061))⁵¹⁰.

We evaluated the neutralization activity of DXP-604 IgG and IgA1 against circulating subvariants BQ.1, BQ.1.1, BA.2.75 and BA.2.75.2, in addition to BA.1, BA.2 and BA.4/5, using pseudovirus assays. Dimeric and secretory IgA1 (IC₅₀: <0.0006 to 0.011 nM) improved the neutralization of BA.1, BA.2 and BA.4/5 Omicron subvariant pseudoviruses by 70.9- to 220-fold compared to monomeric IgG (IC₅₀: 0.091 to 0.836 nM) and by 2.0 to 11.5-fold compared to monomeric IgA1 (IC₅₀: 0.007 to 0.022 nM).

Furthermore, DXP-604 dIgA1 and sIgA1 increased the neutralizing activity against BQ. 1 (IC₅₀: 3.20 and 1.41 nM, by 2.6- and 6.0-fold), BQ.1.1 (IC₅₀: 4.88 and 1.59 nM, by 1.8- to 5.5-fold) and BA.2.75 (IC₅₀: 0.028 and 0.013 nM, by 37.0- and 78.0-fold) compared to IgG. However, DXP-604 IgA1 forms could not restore neutralization activity against BA.2.75.2 and similar results would be expected against XBB and XBB. 1 which carry four additional RBD mutations compared to BA.2.75.2. DXP-604 was recently shown to exhibit no or poor neutralization activity against variants carrying the F486S mutation such as BA.2.75.2 and XBB¹⁰.

Interestingly, 01A05 IgG which showed no binding to BA.2 could neutralize BA.2.75.2 (IC₅₀: 12.08 nM) and the effect was greatly improved using mIgA1 (IC₅₀: 0.067 nM, by 180-fold), dIgA1 (IC₅₀: 0.0165 nM, by 733-fold) or sIgA1 (IC₅₀: 0.006 nM, by 2023-fold). As previously observed with a few other monoclonal antibodies⁴⁹⁻⁵⁰, the R493Q reversion mutation in BA.2.75.2 at least partially restored the 01A05 neutralizing epitope found in ancestral SARS-CoV-2.

Since many Omicron subvariants harbouring multiple convergent mutations are simultaneously circulating in different parts of the world¹⁰, our results suggest that a cocktail of dimeric or secretory IgA1 antibodies, including DXP-604 or 01A05 described here and those newly identified broad neutralizers^{10,51}, would be necessary to neutralize most, if not all, emerging Omicron subvariants, and future VOCs.

### Example 6

### Materials and Methods

### Study Subjects

Study inclusion criteria included subjects who were older than 18 years of age, received inactivated and/or mRNA vaccines with a documented vaccination history (type of vaccine, number of doses, interval between the doses, days after the latest dose, and infection data), and were willing and able to provide written informed consent. The study included 185 samples from 111 healthy volunteers (66% females, median age of 30 years) in Sweden in 2021-2022 who received two or three doses of inactivated vaccine (CoronaVac (Sinovac) or BBIBP-CorV (Sinopharm), 1 to 3 doses of an mRNA vaccine (BNT162b2 (Pfizer-BioNTech) or mRNA-1273 (Moderna) or a combination of both (two doses inactivated vaccine followed by a heterologous mRNA boost), some of whom had experienced BTIs during the Omicron BA. 1 wave. A group receiving one or two doses of mRNA vaccine after SARS-CoV-2 infection was also included. Demographic data of vaccinated individuals are summarized in Table 3.

TABLE 3
Demographic data of vaccinated individuals

| | | | | Median |
|---|---|---|---|---|
| | | | | sampling day |
| | | | Median age | after |
| | | | (IQR)^{a}, | vaccination |
| Groups | Number | Male/Female | years | (IQR) |
| Before vaccination | 7 | 3/4 | 37 (28-38) | |
| Inactivated vaccine | | | | |
| 2^{nd} dose | 5 | 2/3 | 29 (28-32) | 35 (16-70) |
| 3^{rd} dose | 6 | 2/4 | 27.5 (26-35) | 68.5 (38-91) |
| mRNA vaccine | | | | |
| 1^{st} dose | 18 | 7/11 | 35.5 (27-40) | 17.5 (14-21) |
| 2^{nd} dose | 36 | 15/21 | 38.5 (28-51) | 28 (17-52) |
| 3^{rd} dose | 22 | 6/16 | 30 (29-44) | 69 (19- 126) |
| Heterologous vaccine: | 13 | 5/8 | 28 (27-30) | 27 (19-36) |
| Inactivated + 1 dose | | | | |
| mRNA vaccine | | | | |
| Infected + mRNA vaccine | 10 | 6/4 | 43 (32-52) | 30 (17-37) |
| BTI | 22 | 6/16 | 30.5 (27-45) | 38 (18- 138) |

Samples were collected 5-59 days (median day 20) after each mRNA dose including after mRNA heterologous boost, 6-92 days (median day 48) after dose 2 and 3 of inactivated vaccine and 8-43 days (median day 17.5) after a BTI. For a subset of individuals, we also followed the dynamics of antibody response 9 months after the second and third dose of mRNA vaccinations, respectively, and 10 months after BTI in individuals that received 2 or 3 mRNA vaccine doses. Infection was confirmed when an individual tested positive for antigen or qPCR test. Saliva and plasma samples from pre-vaccinated, uninfected healthy donors in our cohort were also collected and used as negative controls. The study was approved by the ethics committee in the institutional review board of Stockholm.

### Detection of Antibodies Specific to SARS-CoV-2 RBD

To assess anti-RBD binding activity, high-binding Corning half-area plates (Corning #3690) were coated with RBD derived from G614, BA.1, BA.2 or BA.4/5 (1.7 μg ml⁻¹) in PBS and incubated overnight at 4° C. Serial dilutions of saliva in PBS with 5% skim milk supplemented with 0.1% Tween 20 were added, and the plates were subsequently incubated for 1.5 hours at room temperature. The plates were then washed and incubated for 1 hour with horseradish peroxidase (HRP)-conjugated goat anti-human IgM (Invitrogen #A18835), goat anti-human IgA (Jackson #109-036-011), or goat anti-human IgG (Invitrogen #A18805) antibodies (all diluted 1:5000 in PBS supplemented with 5% skim milk and 0.1% Tween 20). For detection of secretory immunoglobulins (sIgA and sIgM), plates were incubated for 1 hour with HRP-conjugated goat anti-secretory component antibodies (Nordic-MUbio, #GAHu/SC/PO). The bound antibodies were visualized using tetramethylbenzidine substrate (Sigma #T0440). The colour reaction was stopped with 0.5 M H₂SO₄ after 10 min of incubation, and the absorbance was measured at 450 nm in an ELISA plate reader (Varioskan, Thermo Scientific).

Plasma IgA and IgM and salivary antibody levels are reported as arbitrary units (AU) ml⁻¹ based on a standard curve generated with data derived from a serially diluted highly positive in-house serum pool. Plasma and salivary IgG levels are expressed as binding antibody units (BAU) ml⁻¹ after calibrating in-house standards to the WHO International Standard for anti-SARS-CoV-2 immunoglobulin (NIBSC, 20/136)⁶⁷⁻⁶⁸. For secretory immunoglobulin, serial dilutions of human monoclonal secretory IgA anti-RBD antibodies (DXP-604) were used for the generation of a standard curve and measurement of concentrations (ng ml⁻¹). Salivary IgA anti-RBD antibodies were normalized according to the total level of salivary IgA (AU μg⁻¹ total IgA) to compensate for the different salivary flow rates between individuals. The positive cut-off was calculated to be 2 standard deviations (2SD) higher than the mean of a pool of samples taken from pre-vaccinated and noninfected individuals (n=7).

### Detection of Total IgA Immunoglobulin

To assess total IgA, high-binding Corning half-area plates (Corning #3690) were coated overnight at 4° C. with polyclonal goat anti-human IgA (Southern Biotech, #C5213-R466) (2 g ml⁻¹) in PBS. Serial dilutions of saliva in PBS supplemented with 5% skim milk and 0.1% Tween 20 were added, and the plates were subsequently incubated for 1.5 hours at room temperature. The plates were then washed and incubated with HRP-conjugated polyclonal goat anti-human IgA (Jackson #109-036-011) diluted 1:15000. The bound antibodies were detected as described above. Serial dilutions of human monoclonal IgA were used for the generation of standard curves and measurement of concentrations (ng ml¹).

### Production of SARS-CoV-2 RBD Protein

The RBDs of G614, Alpha, Beta, and Omicron (BA.1, BA.2, BA.4/5) variants were ordered as GeneString from GeneArt (Thermo Fisher Scientific). All sequences of the RBD (aa 319-541 in GenBank: MN908947) were inserted into an NcoI/NotI compatible variant of an OpiE2 expression vector carrying the N-terminal signal peptide of the mouse Ig heavy chain and a C-terminal 6×His-tag. RBD of G614, Beta, Delta and Omicron were expressed in a baculovirus-free expression system in High Five insect cells and purified on HisTrap Excel columns (Cytiva) followed by size-exclusion chromatography on 16/600 Superdex 200-pg columns (Cytiva)^{69,70}.

### Isolation of IgG Antibodies by Sorting RBD-Binding Memory B Cells

The 01A05 IgG antibody was isolated by sorting RBD-binding memory B cells from convalescent patients infected with the G614 strain. The G614 RBD was labelled with either allophycocyanin (APC) or phycoerythrin (PE) for use in a two-fluorescent-dye sorting strategy. XG014³⁷ and DXP-604²³⁵ IgG were previously isolated by sorting RBD-binding memory B cells from convalescent patients infected with the Wuhan strain. rmAb23 was previously isolated using an antibody repertoire prepared by sequencing PBMCs from patients infected with the Wuhan strain followed by matching of the VH3-53-J6 heavy chain with a common IGKV1-9 light chain to produce recombinant antibodies³⁴.

### Cloning of Neutralizing IgG Antibodies to Generate the IgA Forms

The heavy and light chain variable genes of DXP-604, XG014, 01A05 and rmAb23 neutralizing IgG antibodies were cloned into separate pcDNA 3.4 vectors to mediate the fusion to an IgA1 constant region and a light chain constant region gene (kappa for 01A05, rmAb23 and DXP-604 and lambda for XG014), respectively (GenScript). The J-chain and SC genes were cloned into separate pcDNA 3.4 expression plasmids for the assembly of dimeric IgA and secretory IgA.

### Production and Purification of Antibodies

The IgG and IgA1 antibodies 01A05, rmAb23, XG014 and DXP-604 were produced by transfection of HD CHO-S cells with plasmids in a 30-ml volume (GenScript). Monoclonal IgA1 antibodies were produced in CHO cells transiently transfected with two plasmids expressing a heavy and light chain. For the expression of dimeric and secretory IgA1 antibodies, cells were co-transfected with plasmids carrying the J-chain and SC. The IgG and IgA1 antibodies were purified by single-step affinity chromatography using immobilized protein A (MabSelect SuRe™ LX, Cytiva) or anti-IgA antibody (CaptureSelect™ IgA Affinity Matrix), respectively (GenScript).

### Binding of Monoclonal and Recombinant Antibodies Specific to SARS-CoV-2 RBD

To assess the anti-RBD IgG binding activity, high-binding Corning half-area plates (Corning #3690) were coated with RBD derived from G614WT, Beta, Delta and Omicron (1.7 μg ml⁻¹) in PBS and incubated overnight at 4° C.⁶⁸. Serial dilutions of antibody in PBS with 0.1% bovine serum albumin (BSA) were added, and the plates were subsequently incubated for 1.5 hours at room temperature. The plates were then washed and incubated with HRP-conjugated goat anti-human IgG (Invitrogen #A18805) (diluted 1:15000 in 0.1% BSA-PBS) followed by tetramethylbenzidine substrate. For each sample, the EC₅₀ values were calculated using four-parameter nonlinear regression GraphPad Prism 7.04 software⁷¹.

### Pseudovirus Neutralization Assay Based on the VSV Platform

The gene coding for the full S protein of D614G, Alpha, Beta, Gamma, Delta, Kappa, Delta plus, Mu, Lambda, Iota, Omicron (BA.1, BA.2, BA.3, BA.1/BA.2 subvariants BA.1. 1 (BA.1+R346K), BA.2. 12.1 (BA.1+L452Q+S704L) and BA.2. 13 (BA.1+L452M)) and clade 1b SARS-CoV-2 related sarbecoviruses (RaTG13 and Pangolin-GD) were cloned in the pcDNA3. 1 vector. S pseudotyped virus was prepared based on a VSV pseudotyped virus production system. After transfection and culture, the supernatant containing pseudotyped virus was harvested, filtered, diluted to obtain the same particle number across samples, as determined based on quantitative analysis by RT-PCR, and frozen at −80° C. for further use. Pseudovirus neutralization assays were performed using the Huh-7 cell line (Japanese Collection of Research Bioresources [JCRB], 0403) or 293T cells overexpressing human angiotensin-converting enzyme 2, also called 293T-hACE2 cells (Sino Biological Company). Monoclonal antibodies were serially diluted in DMEM (HyClone, SH30243.01) and mixed with pseudovirus in 96-well plates. After the mixture was incubated for 1 hour in a 37° C. incubator with 5% CO2, the digested Huh-7 cells or 293T-hACE2 cells were seeded. After incubation, the supernatant was discarded, D-luciferin reagent (PerkinElmer, 6066769) was added to avoid a light reaction, and the luminescence value was detected with a microplate spectrophotometer (PerkinElmer, Ensight, 6005290). IC50 was determined by a four-parameter logistic regression model.

### Pseudovirus Neutralization Assay Based on the HIV Platform

The human-codon optimized gene coding for the S protein of G614, BA.1, BA.2 and BA.4/5 lacking the C-terminal 19 codons (S_{Δ19}) was synthesized by GenScript. The S_{Δ19} gene of BA.2.75, BA.2.75.2, BQ. 1, and BQ.1.1 was constructed by site-directed mutagenesis (QuikChange Multi Site-Directed Mutagenesis Kit, Agilent) using BA.2 or BA.4/5 S_{Δ19} gene as template. To generate (HIV-1/NanoLuc2AEGFP)-SARS-CoV-2 particles, three plasmids were used, with a reporter vector (pCCNanoLuc2AEGFP), HIV-1 structural/regulatory proteins (pHIV_{NL}GagPol) and SARS-CoV-2 SΔ19 carried by separate plasmids as previously described⁷². 293FT cells were transfected with 7 μg of pHIV_{NL}GagPol, 7 μg of pCCNanoLuc2AEGFP, and 2.5 μg of a pSARS-CoV-2-S_{Δ19} carrying the S_{Δ19} gene from G614 or Omicron variants (at a molar plasmid ratio of 1:1:0.45) using 66 μl of polyethylenimine (PEI).

Tenfold serially diluted monoclonal antibodies were incubated with pseudotyped SARS-CoV-2 virus (G614, BA.1, BA.2, BA.4/5, BA.2.75, BA.2.75.2, BQ.1, and BQ.1.1) for 1 hour at 37° C. The mixture was subsequently incubated with ACE2-expressing HEK293T cells for analyses of G614 or Omicron pseudoviruses for 48 hours, after which the cells were washed with PBS and lysed with Luciferase Cell Culture Lysis reagent (Promega). NanoLuc luciferase activity in the lysates was measured using the Nano-Glo Luciferase Assay System (Promega) with a Tecan Infinite microplate reader. The relative luminescence units were normalized to those derived from cells infected with pseudotyped virus in the absence of monoclonal antibodies. The IC₅₀ values for the monoclonal antibodies were determined using four parameter nonlinear regression (the least squares regression method without weighting; constraints: top, 1; bottom, 0) (GraphPad Prism 7.04 software).

### Microneutralization Assay

The SARS-CoV-2 G614 strain and VOCs (Alpha, Beta, Delta, and Omicron BA. 1, BA.2 and BA.5) were isolated from patients in Pavia, Italy, and identified by next-generation sequencing. The neutralizing activities of the antibodies were determined via microneutralization assays⁷³. Briefly, 50 μl of an antibody, starting at 25 μg ml⁻¹ and increased in a twofold dilution series, was mixed in a flat-bottom tissue culture 96-well microtiter plate (COSTAR, Corning Incorporated) with an equal volume containing a 100 median tissue culture infectious dose (TCID50) of a SARS-CoV-2 strain that had been previously titrated. All dilutions were performed using Eagle's minimum essential medium to which 1% (w/v) penicillin, streptomycin and glutamine and 5 μg ml⁻¹ trypsin had been added. After 1 hour of incubation at 33° C. in 5% CO2, VERO E6 cells (VERO C1008 [Vero 76, clone E6, Vero E6]; ATCC® CRL-1586™) were added to each well. After 3 days of incubation, the cells were stained with Gram's crystal violet solution (Merck) plus 5% formaldehyde (40% m/v) (Carlo Erba S.p.A.) for 30 min. Microtitre plates were then washed in running water. Wells were analysed to evaluate the degree of cytopathic effect compared to untreated controls. Each experiment was performed in triplicate. The IC₅₀ was determined using four-parameter nonlinear regression (GraphPad Prism).

### Virus was Detected by Immunofluorescence Following Neutralizing Antibody Assay

DXP-604 monomeric IgG, and monomeric, dimeric, and secretory IgA1 antibodies, each at a concentration of 3.3 nM were mixed with an equal volume of Omicron BA. 1 (200 plaque-forming units (PFU) per 100 μL) in Dulbecco's modified Eagle's medium (DMEM) supplemented with 2% foetal calf serum (FCS) and gentamycin and incubated at 37° C. for one hour. Next, confluent VeroE6 cells in 96-well plates were washed twice with serum-free DMEM, and the cells were infected with 100 μl of an mAb-virus mix or with virus and no antibodies for 1 hour at 37° C. with 5% CO². The cells were washed twice, and 100 μL of DMEM with 2% FCS and gentamycin was added to each well. After 9 hours of infection, the cells were fixed with 4% formaldehyde overnight. The following day, the cells were washed once with PBS, permeabilized with 0.2% Triton-X in PBS for 15 min at room temperature, and washed. Nonspecific binding was blocked with 3% BSA in PBS at 37° C. for 1 hour. A primary antibody (mouse anti-dsRNA) at a 1:200 dilution with PBS containing 2% BSA was added and incubated for 2 hours at 37° C. After 3 washes, secondary goat anti-mouse Alexa 488 (Jackson ImmunoResearch) in 1:200 in PBS containing 1% BSA and DAPI nuclear stain was added and incubated for 1 hour at 37° C. Finally, 4 washes with PBS were performed, 150 μL of PBS was added to each well, and microscopy was performed using a Leica DMi8 with 20× objectives. The same microscopy settings were used for all images. ImageJ version 2.1.0. was used for processing, and all images were linearly stretched equally. The concentration of each antibody analysed (3.3 nM) corresponded to the IC50 of monomeric IgA antibody.

### Surface Plasmon Resonance (SPR)

Antibody binding properties were analysed at 25° C. using a Biacore 8K instrument (GE Healthcare) with 10 mM HEPES pH 7.4, 150 mM NaCl, 3 mM EDTA, and 0.005% Tween-20 as running buffer. SARS-CoV-2 S-trimers (2, 7, 35 and 70 nM) was immobilized on the surface of a CM5 chip (Cytiva) by standard amine coupling. Increasing concentrations of antibody (3.125, 6.25, 12.5 25, and 50 nM) were injected at a single-cycle kinetics setting (association time: 180 sec, flow rate: 30 μL/min.), and dissociation was followed for 10 min. Analyte responses were corrected for nonspecific binding and buffer responses. Curve fitting and data analysis were performed with Biacore™ Insight Evaluation Software v.2.0. 15. 12933. The fitting of Kon and Koff was performed separately using two different kinetics models. For the Kon, 1:1 binding model was used, while for Koff, 1:1 dissociation model was used.

### Computational Modelling

Computational structure modelling was performed based on binding of 01A05 to RBD of variants (Alpha, Beta, Delta, and Omicron) or through previously published information (Protein Data Bank (PDB) files) describing docking (rmAb23)³⁴ or crystallization (DXP-604 and XG014) studies^{35,36}.

The 01A05 variable fragment was modelled according to the canonical structure method with the program Rosetta Antibody⁷⁴ as previously described⁷⁵. Docking was performed using RosettaDock v3.1 as previously described⁷⁶. In summary, 01A05 model was docked to WT RBD experimental structure (PDB ID: 6m17). Amongst the thousands of computationally generated complexes, the decoy in better agreement with experimental data (competition with hACE2 and differential neutralization activity against SARS-CoV-2 variants) was selected and further refined by computational docking.

The selected models of 01A05 and rmAb23 were subjected to a 350 ns molecular dynamics (MD) simulation to adjust the local geometry and verify that the structure was energetically stable. MD was performed with GROMACS⁷⁷. The system was initially set up and equilibrated through standard MD protocols: proteins were centered in a triclinic box, 0.2 nm from the edge, filled with SPCE water model and 0.15 m Na⁺Cl⁻ ions using the AMBER99SB-ILDN protein force field. Energy minimization was performed in order to let the ions achieve a stable conformation. Temperature and pressure equilibration steps, respectively at 310 K and 1 Bar, of 100 ps each were completed before performing the full MD simulations with the above-mentioned force field. MD trajectory files were analyzed after removal of periodic boundary conditions. The stability of each simulated complex was verified by root mean square deviation and visual analysis.

The structures of mIgG, mIgA and dIgA DXP-604 bound to two SARS-CoV-2 Spike trimers was built using PyMOL software (The PyMOL Molecular Graphics System, Version 2.0 Schrödinger, LLC).

### Quantification and Statistical Analysis

Two-sided Mann-Whitney U test was performed for comparisons of anti-SARS-CoV-2 antibody levels between groups. Correlation analysis between antibody levels was performed using Spearman's rank correlation. All analyses and data plotting were performed with GraphPad 7.05. A p value less than 0.05 was considered to be statistically significant.

### REFERENCES

1. Chen, Y. et al. Emerging SARS-CoV-2 variants: Why, how, and what's next? Cell Insight 1, 100029 (2022).
2. Cao, Y. et al. Omicron escapes the majority of existing SARS-CoV-2 neutralizing antibodies. Nature 602, 657-663 (2022).
3. Tuekprakhon, A. et al. Antibody escape of SARS-CoV-2 Omicron BA.4 and BA.5 from vaccine and BA. 1 serum. Cell 185, 2422-2433.e13 (2022).
4. Viana, R. et al. Rapid epidemic expansion of the SARS-CoV-2 Omicron variant in southern Africa. Nature 603, 679-686 (2022).
5. Wang, Q. et al. Alarming antibody evasion properties of rising SARS-CoV-2 BQ and XBB subvariants. Preprint at https://www.biorxiv.org/content/10. 1101/2022. 11.23.517532v1 (2022).
6. Mannar, D. et al. SARS-CoV-2 Omicron variant: Antibody evasion and cryo-EM structure of spike protein-ACE2 complex. Science 375, 760-764 (2022).
7. Callaway, E. Are COVID surges becoming more predictable? New Omicron variants offer a hint. Nature 605, 204-206 (2022).
8. Callaway, E. COVID ‘variant soup’ is making winter surges hard to predict. Nature 611, 213-214 (2022).
9. Callaway, E. What Omicron's BA.4 and BA.5 variants mean for the pandemic. Nature 606, 848-849 (2022).
10. Cao, Y. et al. Imprinted SARS-CoV-2 humoral immunity induces convergent Omicron RBD evolution. Preprint at https://www.biorxiv.org/content/10. 1101/2022.09. 15.507787v1 (2022).
11. Iketani, S. et al. Antibody evasion properties of SARS-CoV-2 Omicron sublineages. Nature 604, 553-556 (2022).
12. Hirsch, C. et al. SARS-CoV-2-neutralising monoclonal antibodies to prevent COVID-19. Cochrane Database Syst. Rev. 6, CD014945 (2022).
13. Hammarström, L., Abolhassani, H., Baldanti, F., Marcotte, H. & Pan-Hammarström, Q. Development of passive immunity against SARS-CoV-2 for management of immunodeficient patients—a perspective. J. Allergy Clin. Immunol. 146, 58-60 (2020).
14. Hammarström, L., Marcotte, H., Piralla, A., Baldanti, F. & Pan-Hammarström, Q. Antibody therapy for COVID-19. Curr. Opin. Allergy Clin. Immunol. 21, 553-558 (2021).
15. Nguyen, Y. et al. Pre-exposure prophylaxis with tixagevimab and cilgavimab (Evusheld) for COVID-19 among 1112 severely immunocompromised patients. Clin. Microbiol. Infect. 28, 1654.el-1654.e4 (2022).
16. Uraki, R. et al. Characterization of SARS-CoV-2 Omicron BA.4 and BA.5 isolates in rodents. Nature 1-6 (2022) doi:10. 1038/s41586-022-05482-7.
17. Kozlov, M. Omicron's feeble attack on the lungs could make it less dangerous. Nature 601, 177-177 (2022).
18. Russell, M. W., Moldoveanu, Z., Ogra, P. L. & Mestecky, J. Mucosal Immunity in COVID-19: A Neglected but Critical Aspect ofSARS-CoV-2 Infection. Front. Immunol. 11, 611337 (2020).
19. Waltz, E. How nasal-spray vaccines could change the pandemic. Nature 609, 240-242 (2022).
20. Kumar Bharathkar, S. et al. The structures of secretory and dimeric immunoglobulin A. eLife 9, e56098 (2020).
21. Brandtzaeg, P. & Prydz, H. Direct evidence for an integrated function of J chain and secretory component in epithelial transport of immunoglobulins. Nature 311, 71-73 (1984).
22. Woof, J. M. & Russell, M. W. Structure and function relationships in IgA. Mucosal Immunol. 4, 590-597 (2011).
23. Kett, K., Brandtzaeg, P., Radl, J. & Haaijman, J. J. Different subclass distribution of IgA-producing cells in human lymphoid organs and various secretory tissues. J. Immunol. 136, 3631-3635 (1986).
24. Lin, M., Du, L., Brandtzaeg, P. & Pan-Hammarström, Q. IgA subclass switch recombination in human mucosal and systemic immune compartments. Mucosal Immunol. 7, 511-520 (2014).
25. Sterlin, D. et al. IgA dominates the early neutralizing antibody response to SARS-CoV-2. Sci. Transl. Med. 13, eabd2223 (2021).
26. Havervall, S. et al. Anti-Spike Mucosal IgA Protection against SARS-CoV-2 Omicron Infection. N. Engl. J. Med. 387, 1333-1336 (2022).
27. Ejemel, M. et al. A cross-reactive human IgA monoclonal antibody blocks SARS-CoV-2 spike-ACE2 interaction. Nat. Commun. 11, 4198 (2020).
28. Wang, Z. et al. Enhanced SARS-CoV-2 neutralization by dimeric IgA. Sci. Transl. Med. 13, eabf1555 (2021).
29. Planchais, C. et al. Potent human broadly SARS-CoV-2-neutralizing IgA and IgG antibodies effective against Omicron BA. 1 and BA.2. J. Exp. Med. 219, e20220638 (2022).
30. Isho, B. et al. Persistence of serum and saliva antibody responses to SARS-CoV-2 spike antigens in COVID-19 patients. Sci. Immunol. 5, eabe5511 (2020).
31. Huang, N. et al. SARS-CoV-2 infection of the oral cavity and saliva. Nat. Med. 27, 892-903 (2021).
32. Zuo, F., Marcotte, H., Hammarström, L. & Pan-Hammarström, Q. Mucosal IgA against SARS-CoV-2 Omicron Infection. N. Engl. J. Med. 387, e55 (2022).
33. Sheikh-Mohamed, S. et al. Systemic and mucosal IgA responses are variably induced in response to SARS-CoV-2 mRNA vaccination and are associated with protection against subsequent infection. Mucosal Immunol. 15, 799-808 (2022).
34. Yan, Q. et al. Germline IGHV3-53-encoded RBD-targeting neutralizing antibodies are commonly present in the antibody repertoires of COVID-19 patients. Emerg. Microbes Infect. 10, 1097-1111.
35. Du, S. et al. Structurally Resolved SARS-CoV-2 Antibody Shows High Efficacy in Severely Infected Hamsters and Provides a Potent Cocktail Pairing Strategy. Cell 183, 1013-1023.e13 (2020).
36. Zhou, Y. et al. Enhancement versus neutralization by SARS-CoV-2 antibodies from a convalescent donor associates with distinct epitopes on the RBD. Cell Rep. 34, 108699(2021).
37. Liu, Z. et al. An ultrapotent pan-β-coronavirus lineage B (β-CoV-B) neutralizing antibody locks the receptor-binding domain in closed conformation by targeting its conserved epitope. Protein Cell 13, 655-675 (2021).
38. Barnes, C. O. et al. SARS-CoV-2 neutralizing antibody structures inform therapeutic strategies. Nature 588, 682-687 (2020).
39. Cui, Z. et al. Structural and functional characterizations of infectivity and immune evasion of SARS-CoV-2 Omicron. Cell 185, 860-871.e13 (2022).
40. Liu, L. et al. Striking antibody evasion manifested by the Omicron variant of SARS-CoV-2. Nature 602, 676-681 (2022).
41. Boni, M. F. et al. Evolutionary origins of the SARS-CoV-2 sarbecovirus lineage responsible for the COVID-19 pandemic. Nat. Microbiol. 5, 1408-1417 (2020).
42. Chen, R. E. et al. In vivo monoclonal antibody efficacy against SARS-CoV-2 variant strains. Nature 596, 103-108 (2021).
43. Cao, Y. et al. BA.2. 12. 1, BA.4 and BA.5 escape antibodies elicited by Omicron infection. Nature 608, 593-602 (2022).
44. Starr, T. N. et al. Deep Mutational Scanning of SARS-CoV-2 Receptor Binding Domain Reveals Constraints on Folding and ACE2 Binding. Cell 182, 1295-1310.e20 (2020).
45. Ku, Z. et al. Nasal delivery of an IgM offers broad protection from SARS-CoV-2 variants. Nature 595, 718-723 (2021).
46. De Gasparo, R. et al. Bispecific IgG neutralizes SARS-CoV-2 variants and prevents escape in mice. Nature 593, 424-428 (2021).
47. Ke, Z. et al. Structures and distributions of SARS-CoV-2 spike proteins on intact virions. Nature 588, 498-502 (2020).
48. Bachmann, M. F., Mohsen, M. O., Zha, L., Vogel, M. & Speiser, D. E. SARS-CoV-2 structural features may explain limited neutralizing-antibody responses. Npj Vaccines 6, 1-5 (2021).
49. Qu, P. et al. Evasion of neutralizing antibody responses by the SARS-CoV-2 BA.2.75 variant. Cell Host Microbe 30, 1518-1526.e4 (2022).
50. Wang, Q. et al. Antibody evasion by SARS-CoV-2 Omicron subvariants BA.2. 12. 1, BA.4 and BA.5. Nature 608, 603-608 (2022).
51. Bianchini, F. et al. Human neutralizing antibodies to cold linear epitopes and to subdomain 1 of SARS-CoV-2. Preprint at https://www.biorxiv.org/content/10.1101/2022.11.24.515932v1 (2022).
52. Westendorf, K. et al. LY-CoV1404 (bebtelovimab) potently neutralizes SARS-CoV-2 variants. Cell Rep. 39, 110812 (2022).
53. Turelli, P. et al. P2G3 human monoclonal antibody neutralizes SARS-CoV-2 Omicron subvariants including BA.4 and BA.5 and Bebtelovimab escape mutants. Preprint at https://www.biorxiv.org/content/10.1101/2022.07.28.501852v1 (2022).
54. Case, J. B. et al. Resilience of S309 and AZD7442 monoclonal antibody treatments against infection by SARS-CoV-2 Omicron lineage strains. Nat. Commun. 13, 3824 (2022).
55. Li, X. et al. Structural basis of a two-antibody cocktail exhibiting highly potent and broadly neutralizing activities against SARS-CoV-2 variants including diverse Omicron sublineages. Cell Discov. 8, 87 (2022).
56. Gruell, H. et al. SARS-CoV-2 Omicron sublineages exhibit distinct antibody escape patterns. Cell Host Microbe 30, 1-11 (2022).
57. Luo, S. et al. An antibody from single human VH-rearranging mouse neutralizes all SARS-CoV-2 variants through BA.5 by inhibiting membrane fusion. Sci. Immunol. 7, eadd5446 (2022).
58. Su, F., Patel, G. B., Hu, S. & Chen, W. Induction of mucosal immunity through systemic immunization: Phantom or reality? Hum. Vaccines Immunother. 12, 1070-1079 (2016).
59. Sano, K. et al. SARS-CoV-2 vaccination induces mucosal antibody responses in previously infected individuals. Nat. Commun. 13, 5135 (2022).
60. Joo, H. M., He, Y., Sundararajan, A., Huan, L. & Sangster, M. Y. Quantitative analysis of influenza virus-specific B cell memory generated by different routes of inactivated virus vaccination. Vaccine 28, 2186-2194 (2010).
61. Mao, T. et al. Unadjuvanted intranasal spike vaccine elicits protective mucosal immunity against sarbecoviruses. Science 378, eabo2523 (2022).
62. Bonner, A., Furtado, P. B., Almogren, A., Kerr, M. A. & Perkins, S. J. Implications of the Near-Planar Solution Structure of Human Myeloma Dimeric IgA1 for Mucosal Immunity and IgA Nephropathy. J. Immunol. 180, 1008-1018 (2008).
63. Bonner, A., Almogren, A., Furtado, P. B., Kerr, M. A. & Perkins, S. J. The Nonplanar Secretory IgA2 and Near Planar Secretory IgA1 Solution Structures Rationalize Their Different Mucosal Immune Responses. J. Biol. Chem. 284, 5077-5087 (2009).
64. Natarajan, A. et al. Gastrointestinal symptoms and fecal shedding of SARS-CoV-2 RNA suggest prolonged gastrointestinal infection. Med 3, 371-387.e9 (2022).
65. Phalipon, A. et al. Secretory Component: A New Role in Secretory IgA-Mediated Immune Exclusion In Vivo. Immunity 17, 107-115 (2002).
66. Teh, A. Y.-H. et al. Investigation of a monoclonal antibody against enterotoxigenic Escherichia coli, expressed as secretory IgA1 and IgA2 in plants. Gut Microbes 13, 1859813 (2021).
67. Marcotte, H. et al. Immunity to SARS-CoV-2 up to 15 months after infection. iScience 25, 103743 (2022).
68. Sherina, N. et al. Persistence of SARS-CoV-2-specific B and T cell responses in convalescent COVID-19 patients 6-8 months after the infection. Med 2, 281-295.e4 (2021).
69. Bertoglio, F. et al. A SARS-CoV-2 neutralizing antibody selected from COVID-19 patients binds to the ACE2-RBD interface and is tolerant to most known RBD mutations. Cell Rep. 36, 109433 (2021).
70. Korn, J. et al. Baculovirus-free insect cell expression system for high yield antibody and antigen production. Sci. Rep. 10, 21393 (2020).
71. Zuo, F. et al. Heterologous immunization with inactivated vaccine followed by mRNA-booster elicits strong immunity against SARS-CoV-2 Omicron variant. Nat. Commun. 13, 2670 (2022).
72. Schmidt, F. et al. Measuring SARS-CoV-2 neutralizing antibody activity using pseudotyped and chimeric virusesSARS-CoV-2 neutralizing antibody activity. J. Exp. Med. 217, e20201181 (2020).
73. Percivalle, E. et al. Prevalence of SARS-CoV-2 specific neutralising antibodies in blood donors from the Lodi Red Zone in Lombardy, Italy, as at 6 Apr. 2020. Eurosurveillance 25, 2001031 (2020).
74. Sircar, A., Kim, E. T. & Gray, J. J. RosettaAntibody: antibody variable region homology modeling server. Nucleic Acids Res. 37, W474-W479 (2009).
75. Pedotti, M., Simonelli, L., Livoti, E. & Varani, L. Computational Docking of Antibody-Antigen Complexes, Opportunities and Pitfalls Illustrated by Influenza Hemagglutinin. Int. J. Mol. Sci. 12, 226-251 (2011).
76. Simonelli, L. et al. Rapid Structural Characterization of Human Antibody-Antigen Complexes through Experimentally Validated Computational Docking. J. Mol. Biol. 396, 1491-1507 (2010).
77. Van Der Spoel, D. et al. GROMACS: Fast, flexible, and free. J. Comput. Chem. 26, 1701-1718 (2005).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for treating infection of a SARS-CoV-2 Omicron variant in a subject in need thereof, the method comprising:
administering to the subject a pharmaceutical composition comprising an IgA antibody which binds to and neutralizes SARS-CoV-2 Omicron variant, the IgA comprising (a) a heavy chain comprising the amino acid sequence of SEQ ID NO: 1, and (b) a light chain comprising the amino acid sequence of SEQ ID NO: 2.

2. The method of claim 1, wherein the IgA antibody is a monomeric IgA1 antibody.

3. The method of claim 1, wherein the IgA antibody is a dimeric IgA1 antibody.

4. The method of claim 1, wherein the IgA antibody is a secretory IgA1 antibody.

5. The method of claim 1, wherein the IgA antibody further comprises a J chain comprising the amino acid sequence of SEQ ID NO: 3.

6. The method of claim 1, wherein the IgA antibody further comprises a secretory component comprising the amino acid sequence of SEQ ID NO: 4.

7. The method of claim 1, wherein the SARS-CoV-2 Omicron variant is BA.1, BA.2 or BA.5.

8. The method of claim 1, wherein the pharmaceutical composition further comprises a pharmaceutically acceptable excipient, carrier, or diluent.

9. The method of claim 1, wherein the pharmaceutical composition is administered via intranasal.

10. The method of claim 1, wherein the pharmaceutical composition further comprises a second antibody which binds to and neutralizes a SARS-CoV-2 Omicron variant.
